SEQUENCE LISTING (17 sequences)

SEQ ID NO: 1 - protein, 4 aa, synthetic construct
  REGION 1..4: note = peptide moiety (P) of Formula I
LDPR

SEQ ID NO: 2 - protein, 5 aa, synthetic construct
  REGION 1..5: note = peptide moiety (P) of Formula I
TLDPR

SEQ ID NO: 3 - protein, 6 aa, synthetic construct
  REGION 1..6: note = peptide moiety (P) of Formula I
ATLDPR

SEQ ID NO: 4 - protein, 7 aa, synthetic construct
  REGION 1..7: note = peptide moiety (P) of Formula I
NATLDPR

SEQ ID NO: 5 - protein, 8 aa, synthetic construct
  REGION 1..8: note = peptide moiety (P) of Formula I
TNATLDPR

SEQ ID NO: 6 - protein, 9 aa, synthetic construct
  REGION 1..9: note = peptide moiety (P) of Formula I
ATNATLDPR

SEQ ID NO: 7 - protein, 10 aa, synthetic construct
  REGION 1..10: note = peptide moiety (P) of Formula I
KATNATLDPR

SEQ ID NO: 8 - protein, 11 aa, synthetic construct
  REGION 1..11: note = peptide moiety (P) of Formula I
SKATNATLDPR

SEQ ID NO: 9 - protein, 12 aa, synthetic construct
  REGION 1..12: note = peptide moiety (P) of Formula I
ESKATNATLDPR

SEQ ID NO: 10 - protein, 13 aa, synthetic construct
  REGION 1..13: note = thrombin-recognition site in PAR-1
PESKATNATLDPR

SEQ ID NO: 11 - protein, 14 aa, synthetic construct
  REGION 1..14: note = peptide moiety (P) of Formula I
RPESKATNATLDPR

SEQ ID NO: 12 - protein, 15 aa, synthetic construct
  REGION 1..15: note = peptide moiety (P) of Formula I
RRPESKATNATLDPR

SEQ ID NO: 13 - protein, 16 aa, synthetic construct
  REGION 1..16: note = peptide moiety (P) of Formula I
ARRPESKATNATLDPR

SEQ ID NO: 14 - protein, 17 aa, synthetic construct
  REGION 1..17: note = peptide moiety (P) of Formula I
RARRPESKATNATLDPR

SEQ ID NO: 15 - protein, 18 aa, synthetic construct
  REGION 1..18: note = peptide moiety (P) of Formula I
TRARRPESKATNATLDPR

SEQ ID NO: 16 - protein, 19 aa, synthetic construct
  REGION 1..19: note = peptide moiety (P) of Formula I
RTRARRPESKATNATLDPR

SEQ ID NO: 17 - protein, 20 aa, synthetic construct
  REGION 1..20: note = peptide moiety (P) of Formula I
ARTRARRPESKATNATLDPR

(19) United States
(12) Patent Application Publication
(10) Pub. No.: US 2023/0355727 A1
(43) Pub. Date: Nov. 9, 2023

(54) TREATMENT OF RETINAL DISORDERS

(71) Applicant: Tel HaShomer Medical Research Infrastructure and Services Ltd., Ramat-Gan (IL)

(72) Inventors: Joab CHAPMAN, Kiryat-Ono (IL); Efrat SHAVIT STEIN, Karkur (IL); Ygal ROTENSTREICH, Kfar-Bilu (IL); Ifat SHER ROSENTHAL, Shoham (IL)

(73) Assignee: Tel HaShomer Medical Research Infrastructure and Services Ltd., Ramat-Gan (IL)

(21) Appl. No.: 18/144,968

(22) Filed: May 9, 2023

Related U.S. Application Data
(63) Continuation of application No. PCT/IL2021/051330, filed on Nov. 9, 2021.
(60) Provisional application No. 63/111,151, filed on Nov. 9, 2020.

(51) Int. Cl. A61P 27/02; A61K 38/55; A61K 9/00

(52) U.S. Cl. CPC A61K 38/55; A61K 9/0048; A61P 27/02

(57) ABSTRACT

Ophthalmic formulations comprising a PAR1 antagonist and/or an agent that interferes with an interaction of PAR1 and a protease (e.g., thrombin), and an ophthalmically acceptable carrier, and uses thereof in treating or preventing retinal pathologies such as retinal degeneration, are provided. The agent can be, for example, a peptide conjugate represented by Formula I, as defined in the specification.

### RELATED APPLICATIONS

This application is a Continuation of PCT Pat. Application No. PCT/IL2021/051330 having International filing date of Nov. 9, 2021, which claims the benefit of priority under 35 USC §119(e) of U.S. Provisional Pat. Application No. 63/111,151 filed on Nov. 9, 2020. The contents of the above applications are all incorporated by reference as if fully set forth herein in their entirety.

### SEQUENCE LISTING STATEMENT

The XML file, entitled 96213SequenceListing.xml, created on May 9, 2023, comprising 22,213 bytes, submitted concurrently with the filing of this application is incorporated herein by reference.

### FIELD AND BACKGROUND OF THE INVENTION

The present invention, in some embodiments thereof, relates to therapy and, more particularly, but not exclusively, to novel ophthalmic formulations and methods utilizing same for treating retinal diseases and disorders, for example, diseases and disorders associated with retinal degeneration.

Diabetic retinopathy (DR) is the leading ocular complication of diabetic type I and a leading cause of sight-loss among the working age population of industrialized regions. In 2010 it was estimated that DR affected over 100 million patients worldwide and these estimates are expected to rise to over 190 million by 2030. Diabetic macular edema (DME) and proliferative diabetic retinopathy (PDR) are the major sight-threatening complications of diabetes. In addition, diabetic ischemic maculopathy involving retinal microvascular degeneration within the macular region can also result in loss of central visual acuity. These diseases are associated with poor glycemic control and prolonged disease duration.

The prolonged exposure to the diabetic milieu leads to the activation of a number of interconnecting biochemical pathways that contribute to DR pathology. A complex interplay between neuroglial and vascular damage results from hyper-glycaemia-induced metabolic stress. From the microvascular perspective, hypo-perfusion early in the disease due to loss of the cells making up the endothelium ultimately leads to compensatory growth of new fragile and leaky blood vessels.

The major angiogenic factor in PDR that promotes neovascularization and vascular leakage is Vascular Endothelial Growth Factor (VEGF). Compromise of the blood retina barrier integrity leads to the extravasation of fluid and inflammatory mediators, creating sight threatening edema and exacerbating inflammatory conditions. The concurrent or preceding neuro-glial dysfunction perpetuates the retinal damage.

A major feature of diabetic tissue is the overactivation of coagulation, partly due to the inhibition of plamin, the major fibrinolytic enzyme. This is highly relevant for the inflammatory and neovascularization pathologies associated with diabetic retinopathy and is also relevant for any other pathology or medical condition that involves retinal inflammatory and/or neovascularization.

Current treatments target the microvascular changes associated with advanced stages of the disease, are highly invasive, do not prevent the damage to the neuroretina, and require trained ophthalmologists or retinal specialists that are not available for all populations.

Although diabetic retinopathy was traditionally considered a microvascular complication of diabetes, the chronic hyperglycemia in diabetic patients induces retinal inflammation and oxidative stress that further impair the function of retinal neurons and glial cells.

Retinal degeneration diseases such as age-related macular degeneration (AMD) and retinitis pigmentosa (RP) are also a leading cause of blindness worldwide. AMD affects mostly people age 65 and up. With the aging of the population, the prevalence of AMD is predicted to increase sharply reaching 288 million in 2040. Retinitis pigmentosa (RP) is a group of incurable hereditary retinal degeneration diseases that affects nearly 2 million patients worldwide and is characterized by progressive degeneration of rod and cone photoreceptors. Recently gene therapy was approved for one of the 80 causative genes of RP, but it is beneficial for few RP patients who carry mutations in that gene. These diseases are highly heterogeneous with dozens of known causative genes. Many patients cannot be genetically diagnosed and disease progression varies between individuals regardless of the affected gene.

Protease-activated receptor-1 (PAR1) is a G-protein coupled receptor. This receptor carries its own ligand, which remains silent until the serine protease thrombin and other proteases cleave at a specific site within the extracellular N-terminus, exposing a new N-terminal-tethered ligand domain that binds and activates the cleaved receptor. Additional proteases have been found to cleave and activate PAR1, some of them, such as plasmin and Factor Xa cleave at the same site as thrombin and some, such as activated protein C (aPC) and the zinc-dependent matrix metalloproteinase-1 (MMP-1), cleave at other PAR1 activation sites.

Signal transduction initiated by PAR1 activation has many downstream consequences, leading to changes in cellular morphology, proliferation, migration, and adhesion. It was recently shown that selective proteolytic activation of PAR1 by thrombin and MMP-1, plays a central role in enhancing both angiogenesis and tumor growth [Zigler et al. Cancer Res. 2011;71(21):6561-6566]. In addition, activation of PAR1 leads to the synthesis and secretion of functional VEGF protein and that PAR1-induced angiogenesis is mediated by VEGF [Yin et al. FASEB J. 2003;17(2):163-174].

PAR1 and its homologue receptor PAR2 where found to be highly expressed in the neuro-retina, where it mediates calcium signaling, and is upregulated following optic nerve crush injury [Luo et al. Brain Res. 2005;1047(2):159-167].

PAR1 was found to play a functional role in controlling nerve conduction. PAR1 activation was shown to affect the glia component of the node of Ranvier in the peripheral nervous system, causing nerve conduction block [Shavit et al. Brain. 2008;131(Pt 4):1113-1122]. In the central nervous system, PAR1 activation was shown to modulate synaptic transmission by causing LTP and seizure-like activity and potentiates the synaptic NMDA receptor [Maggio et al. J Neurosci. 2008;28(3):732-736; Traynelis and Trejo. Curr Opin Hematol. 2007;14(3):230-235].

Recent studies have suggested that PAR1, thrombin, and MMP-1 are expressed within the ocular microenvironment of patients with PDR and showed that PAR1 and thrombin might facilitate angiogenesis and progression of PDR by inducing endothelial cell migration, as well as secretion of angiogenic mediators [Abu El-Asrar et al. Curr Eye Res. 2016;41(12):1590-1600].

Some of the present inventors have previously reported on findings that support the role of PAR1 activation in mediating neurological dysfunction in diabetic patients [Shavit-Stein et al. PLoS One. 2019;14(7):e0219453]. Thrombin-like activity was elevated in sciatic nerves derived from animal models of streptozotocine-(STZ)-induced diabetes. A significant decrease in PAR1 level was found together with increased physiological thrombin-inhibitors (PN-1, PN-2) indicating that the natural response of the nervous system in these diseases is to increase thrombin inhibition. In addition, a specific thrombin inhibitor, Nα-(2-naphthyl-sulphonyl-glycyl)-DL-p-amidinophenylalanyl-piperidine (NAPAP), was found to prevent the decreased nerve conduction velocity found in diabetic rats. An increased thrombin activity and decreased PAR1 levels were found also in brains of ALS animal model SOD-1. A relatively general thrombin inhibitor, N-alpha-tosyl-L-lysine chloromethyl ketone (TLCK), as well as the PAR1 antagonist SCH79797, were found to increase the life span of these ALS model animals significantly.

WO 2015/173802 discloses a peptide conjugate comprising an alpha-amino protecting moiety, a peptide comprising an amino acid sequence at least 3 amino-acid long derived from the C -terminus of PAR 1, or an active variant thereof, and a protease-disabling moiety, which is usable in treating diseases and disorders associated with excessive PAR1 activity. Studies showing the effect of an exemplary such conjugate on GBM are described in Shavit-Stein et al. (2018) Front. Neurol. 9, 108.

Additional background art includes U.S. Pat. Application Publication Nos. 2009/0281100 and 2004/0092535; and Bastiaans et al. (2013) Graefes Arch. Clin. Exp. Ophthalmol. 251, 1723-1733.

### SUMMARY OF THE INVENTION

According to an aspect of some embodiments of the present invention there is provided an ophthalmic formulation comprising a PAR1 antagonist and/or an agent that interferes with an interaction of PAR1 and a protease, and an ophthalmically acceptable carrier.

According to some of any of the embodiments described herein, the agent is represented by Formula I:

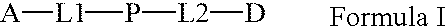

wherein: P is a peptide of at least 3 amino acid residues, comprising or consisting of the amino acid sequence Asp-Pro-Arg; A is an N-terminus protecting group; D is a group capable of interfering with a PAR1/protease interaction; and L1 and L2 are each independently a linking moiety or absent, and a pharmaceutically acceptable carrier, the formulation being for topical application of the agent to an eye of a subject in need thereof.

According to some of any of the embodiments described herein, P in Formula I consists of the amino acid sequence Asp-Pro-Arg.

According to some of any of the embodiments described herein, P in Formula I has 5 amino acid residues.

According to some of any of the embodiments described herein, P in Formula I has an amino acid sequence as set forth in SEQ ID NO:2 (TLDPR).

According to some of any of the embodiments described herein, P in Formula I has an amino acid sequence selected from the amino acid sequences as set forth in SEQ ID NOS:1-17.

According to some of any of the embodiments described herein, A in Formula I is an aromatic N-terminus protecting group.

According to some of any of the embodiments described herein, A in Formula I is tosyl.

According to some of any of the embodiments described herein, D in Formula I is a protease inhibitor.

According to some of any of the embodiments described herein, D in Formula I is a thrombin inhibitor.

According to some of any of the embodiments described herein, D in Formula I is or comprises an acetyl group.

According to some of any of the embodiments described herein, D in Formula I is or comprises chloromethyl ketone.

According to some of any of the embodiments described herein, each of L1 and L2 is absent.

According to some of any of the embodiments described herein, A in Formula I is tosyl and D is or comprises chloromethylketone.

According to some of any of the embodiments described herein, the ophthalmic formulation is configured for topical application to an eye of a subject.

According to some of any of the embodiments described herein, the formulation is in a form of a solution, a gel, an aerosol, a spray, a foam, a mousse, an ointment, a paste, a lotion, a gauze, a wound dressing, a suspension, an adhesive bandage, a non-adhesive bandage, a wipe, a gauze, a pad, and a sponge.

According to some of any of the embodiments described herein, the formulation is in a form of an aqueous solution.

According to some of any of the embodiments described herein, the ophthalmic formulation further comprises one or more of anti-irritants, anti-foaming agents, humectants, deodorants, antiperspirants, preservatives, emulsifiers, occlusive agents, emollients, thickeners, penetration enhancers, colorants, propellants, surfactants, tonicity adjusting agents, disinfecting agents, anti-oxidants, and stabilizers such as a cyclodextrin.

According to some of any of the embodiments described herein, a concentration of said PAR1 antagonist and/or said agent in the formulation is lower than 500 millimolar, or lower than 100 millimolar, or lower than 1 millimolar, or lower than 500 nanomolar, or lower than 100 nanomolar, or lower than 1 nanomolar, and in some embodiments it can be, for example, in a range of from 1 picomolar to 500 millimolar, including any intermediate values and subranges therebetween.

According to an aspect of some embodiments of the present invention there is provided an ophthalmic formulation as described herein in any of the respective embodiments and any combination thereof, for use in treating a disease or disorder associated with overexpression and/or overactivity of PAR1 in a retinal tissue of a subject.

According to an aspect of some embodiments of the present invention there is provided an ophthalmic formulation as described herein in any of the respective embodiments and any combination thereof, for use in treating a disease or disorder treatable by interfering with a PAR1/protease interaction in a retinal tissue of a subject.

According to some of any of the embodiments described herein, the disease or disorder is selected from retinal degeneration, retinal dystrophy, retinal inflammation and abnormal proliferation in the retinal tissue.

According to an aspect of some embodiments of the present invention there is provided an ophthalmic formulation as described herein in any of the respective embodiments and any combination thereof, for use in treating or preventing retinal degeneration in a subject in need thereof.

According to some of any of the embodiments described herein, the retinal degeneration is associated with diabetic retinal neuropathy.

According to some of any of the embodiments described herein, the treatment comprises topical administration of the formulation to, or contacting the formulation with, an eye of the subject.

According to some of any of the embodiments described herein, the treatment comprises topically administering the formulation to the eye of the subject from 1 to 5 or from 1 to 4 times per day.

According to an aspect of some embodiments of the present invention there is provided an article-of-manufacturing comprising the ophthalmic formulation as described herein in any of the respective embodiments and any combination thereof, and means for topically administering the formulation to, or contacting the formulation with, an eye of a subject.

According to some of any of the embodiments described herein, the article-of-manufacturing comprises a container for housing the formulation and means for dispensing the formulation to, or for contacting the formulation with, an eye of the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic representation of the design of selective PAR1 molecules based on the thrombin recognition site sequence in PAR1, as described, for example, in WO 2015/173802.

FIG. 2 presents comparative plots showing retinal function, as measured by ERG, in control C57BL/6 mice (diamonds) and in STZ diabetes-induced mice (triangles). Maximal ERG a-wave response, reflecting rod photoreceptor function, was measured in response to increasing light intensities (Luminance).

FIGS. 3A-3G present confocal microscopy images showing PAR1 expression in the neuroretina in control C57BL/6 mice (FIG. 3A), in diabetic mice (following STZ diabetic induction, FIG. 3B), in retinal sections incubated only with secondary antibody (W/O 1^{st} Ab, FIG. 3C), in retinal sections from PAR1 knockout mice (FIG. 3D), and confocal images of the rod inner and outer retinal segments, showing of PAR1 (in red; FIG. 3E), rhodopsin (in green; FIG. 3F), and a merged image showing co-localization of the two proteins in yellow (FIG. 3G).

FIGS. 4A-4B present the thrombin activity in the posterior segment (FIG. 4A; p=0.03), and the lower retinal function (FIG. 4B, p=0.033) as measured by ERG, in STZ-induced diabetic mice compared to control non-diabetic mice.

FIG. 5 is a bar graph showing the effect of a 7-day treatment (IP injection (0.5 µl/gram, 1/day) and eye drops (10 µl/eye, 1/day)] with either sham (PBS, n=5) or PARIN5 (100 nM, n=7) on retinal function, as measured by ERG (b-wave), in STZ-induced diabetic C57BL/6 mice.

FIG. 6 is a bar graph showing retinal function at 2 and 5 weeks following STZ injection, as measured by ERG (b-wave), in control, non-diabetic mice, in STZ-induced diabetic mice non-treated, and in STZ-induced diabetic mice, treated for 5 weeks daily with eye drops containing PARIN5 (100 nM, all p>0.3).

FIGS. 7A-7F present confocal microscopy images showing PAR1 expression in human retinal sections (FIGS. 7D-F). No staining is observed in human retinal sections incubated only with secondary antibody (W/O 1^{st} Ab, FIGS. 7A-C), supporting the staining specificity for the PAR1 protein in the human neuro-retina. FIGS. 7A and 7D show PAR1 in red; FIGS. 7B and 7E show nuclei in blue; and FIGS. 7C and 7F show merge images.

FIGS. 8A-B present Western blot analysis of antibody staining in mouse retina, optic nerve and brain, in human platelets (PLT), in mouse platelets (PLT) and in retinas from PAR1^{-/-} mice (FIG. 8A) and of PAR1 antibody (NBP-71770, Nuvos biologicals) staining in mouse retina, mouse platelets (PLT) and in retinas from PAR1^{-/-} mice (FIG. 8B).

FIGS. 9A-E present confocal microscopy images showing PAR1 expression in the neuroretina in C57BL/6J mice under physiological conditions. Paraffin retinal cross sections derived from C57BL/6J mice (FIGS. 9A-C and 9E, n=12) and PAR1^{-/-} mice (FIG. 9D, n=2) were stained with anti-PAR1 antibody (red, FIGS. 9A, 9C, and 9D) or secondary antibody only as control (FIG. 9E). In blue are counter-stained with 4′,6-diamidino-2-phenylindole (DAPI, FIGS. 9B and 9D-insert). Index: GCL-ganglion cell layer, IPL-inner plexiform layer, INL-Inner Nuclear Layer; OPL-outer plexiform layer, ONL-Outer Nuclear Layer, IS-(photoreceptor) inner segment, OS-(photoreceptor) outer segment. Images were obtained with a confocal microscope (LSM700). Scale bars: 50 µm.

FIGS. 9F-G present confocal microscopy images showing PAR1 expression pattern in the neuroretina in PFA-perfused (FIG. 9F) vs. non-perfused mice (FIG. 9G), upon staining with anti-PAR1 antibody. Images were obtained with confocal microscope LSM800. GCL-ganglion cell layer, IPL- inner plexiform layer, INL- Inner Nuclear Layer; OPL- outer plexiform layer, ONL-Outer Nuclear Layer, IS- (photoreceptor) inner segment, OS- (photoreceptor) outer segment.

FIGS. 10A-D present confocal microscopy images showing PAR1 co-localization with rhodopsin in mouse retina. Paraffin retinal sections from C57BL/6J mice were co-stained with anti-PAR1 (red) and anti-rhodopsin (green) antibodies. Nuclei were counter-stained with DAPI (blue, FIG. 10A); and magnification of the area defined by the white rectangle in FIG. 10A, in the red (PAR1, FIG. 10B), green (rhodopsin, FIG. 10C) and merged image (FIG. 10D). Index: ONL- Outer Nuclear Layer, OS- (photoreceptor) outer segments.

FIGS. 10E-F show co-localization analysis, using ZEN software (Leica), calculating Pearson’s Correlation Coefficient between the red (PAR1) and green (rhodopsin) channels (FIG. 10E) was 0.92 ± 0.02, indicating a strong overlap between of PAR1 and rhodopsin. Co-localization analysis in the photoreceptor outer segments was performed in three areas in retinal sections derived from two mice (FIG. 10F).

FIGS. 11A-H present confocal microscopy images showing that PAR1 does not co-localize with cone L/M-opsin and S-opsin in mouse retina. Paraffin cross sections of retinas derived from C57BL/6J mice are stained with anti-PAR1 (red in FIGS. 11A, 11E, 11C, and 11G), L/M opsin (green in FIGS. 11B and 11C), and S opsin (green in FIGS. 11F and 11G) antibodies. Nuclei were counter-stained with DAPI (blue in FIGS. 11C and 11G). Images were obtained with confocal microscope LSM800. Index: ONL-Outer Nuclear Layer, OS-(photoreceptor) outer segments.

FIGS. 11D and 11H present the co-localization analysis performed on cone photoreceptor outer segments at three areas using ZEN software (Leica), calculating Pearson’s Correlation Coefficient between the red (PAR1) and green (rhodopsin) channels (FIG. 11D) showed a very low correlation between PAR1 and M/L- and S-opsin staining with Pearson Correlation Coefficients of 0.11 ± 0.110 and 0.04 ± 0.01, respectively. Co-localization analysis in the photoreceptor outer segments was performed in three areas in retinal sections (FIG. 11H). Scale bar: 25 µm.

FIG. 12 is a bar graph showing mRNA expression of the coagulation factors PAR1, Factor X (FX), and prothrombin in the mouse neuroretina, as determined by quantitative real-time reverse transcriptase PCR (qRT-PCR) in six mice (13 week old, P91). Results are presented relative to HPRT using the 2^ΔCT calculating method, and support the expression of the PAR/Thrombin pathway in the neuroretina

FIG. 13 is a bar graph showing thrombin activity in isolated neuroretinas ex-vivo under low and high KCl concentrations. Neuroretinas derived from C57BL/6J mice were incubated ex-vivo in a buffer containing high (56 mM, n=10) or low (5.6 mM, n=10) KCl concentration, and thrombin activity was measured. * p<0.05.

FIGS. 14A-D present the data obtained in immunostaining assays of paraffin retinal sections of non-diabetic (FIGS. 14A-B) and 5-week diabetic (FIGS. 14C-D) C57BL/6 mice stained with anti-PAR1 antibody (red) and counter-stained with DAPI (blue). Images were obtained with confocal microscope LSM800. GCL-ganglion cell layer, IPL-inner plexiform layer, INL-Inner Nuclear Layer; OPL-outer plexiform layer, ONL-Outer Nuclear Layer, IS-(photoreceptor) inner segment, OS-(photoreceptor) outer segment.

FIGS. 15A-B present the data obtained in immunostaining assays of paraffin retinal sections of STZ induced diabetic mice 5 weeks following diabetes induction, Mice perfused with paraformaldehyde before eye removal (FIG. 15A), or non-perfused diabetic mice (FIG. 15B) were stained with anti-PAR1 antibody (red). Images were obtained with confocal microscope LSM800. GCL-ganglion cell layer, IPL-inner plexiform layer, INL-Inner Nuclear Layer; OPL-outer plexiform layer, ONL-Outer Nuclear Layer, IS-(photoreceptor) inner segment, OS-(photoreceptor) outer segment.

FIG. 16 is a graph presenting the comparative thrombin activity in isolated neuroretinas of WT C57BL/6J (n=6) mice (circles) and of RPE65/rd12 (n=6) mice (squares), in the presence and absence of PARIN5 (100 nM).

### DESCRIPTION OF SPECIFIC EMBODIMENTS OF THE INVENTION

The present invention, in some embodiments thereof, relates to therapy and, more particularly, but not exclusively, to novel ophthalmic formulations and methods utilizing same for treating retinal diseases and disorders, for example, diseases and disorders associated with retinal degeneration.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

As discussed in the Background section hereinabove, at present that are no methodologies for early, noninvasive, and easily accessible interventions for treating or preventing retinal degeneration so as to avoid vision loss.

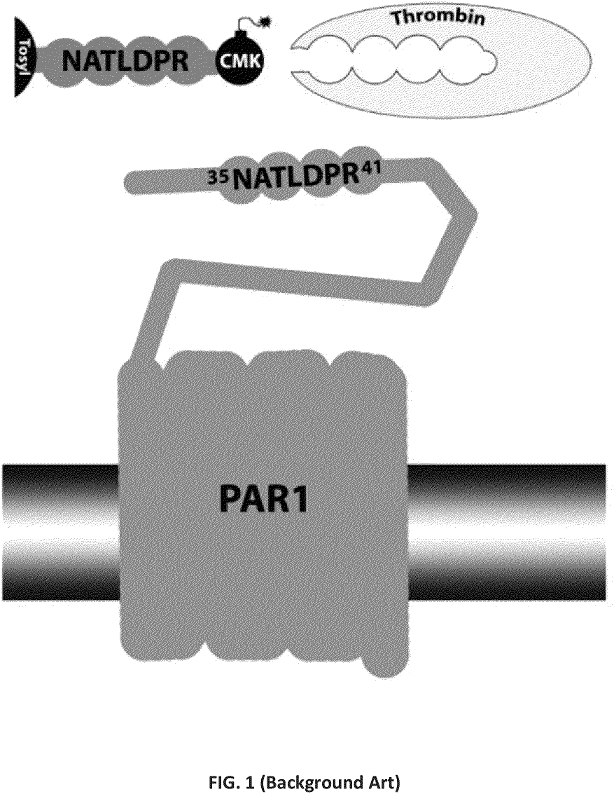

As further discussed in the Background section, the present inventors have designed and successfully practiced unique molecules (peptide conjugates), which are based on the specific thrombin-recognition site in PAR1 (the sequence PESKATNATLDPR; SEQ ID NO: 10) that specifically block the interaction of thrombin and PAR1. See, Background Art FIG. 1. In preliminary experiments, the molecules were screened in-vitro for their ability to inhibit commercially available target proteases such as human and bovine thrombin and thrombin-like activity generated in glioma cell-lines. The molecules were also screened for the major potential side effect related to their ability to inhibit coagulation and the associated risk of hemorrhage. Following these screens, a leading molecule was selected, containing 5 amino-acids (³⁷TLDPR⁴¹ SEQ ID NO:2)-chloromethylketone, designated PARIN5) that presented a significant inhibition of glioma edema volume growth and protection of nerve function in diabetic neuropathy in vivo, suggesting that the PAR1/thrombin signaling axis may present a new avenue for therapeutic intervention for PAR1 associated diseases such as diabetic retinopathy, potentially targeting the pathological angiogenic and neurodegeneration processes.

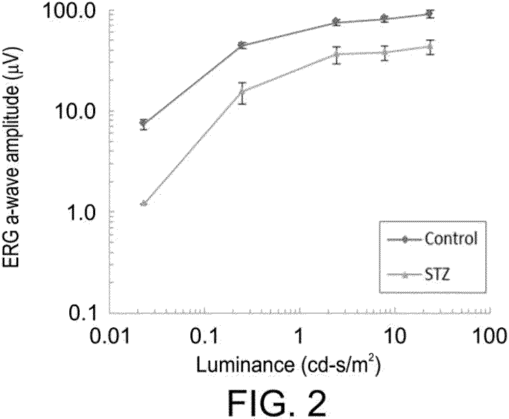

As shown in FIGS. 2, 3A-3G and 4A-4B, 7A-7F, 8A-B, 9A-G, 10A-F, 11A-H, 12, 13, 14A-D, and 15A-B, the present inventors have now uncovered that PAR1 is expressed in the inner and outer layers of the neuroretina in humans and mice and that its expression, as well as thrombin activity, is elevated in the neuroretina of non-diabetic diabetic mice and humans.

These findings indicate that ameliorating retinal degeneration and/or treating conditions associated with PAR1 overexpression in the neuroretina (conditions treatable by inhibiting the PAR1 pathway) can be performed via targeting PAR1/Thrombin pathway and this can be practiced simply by means of ophthalmic administration. This treatment should slow down or even prevent vision loss in patients with retinal degeneration, including patients suffering from conditions such as DR, RP, night blindness and AMD patients.

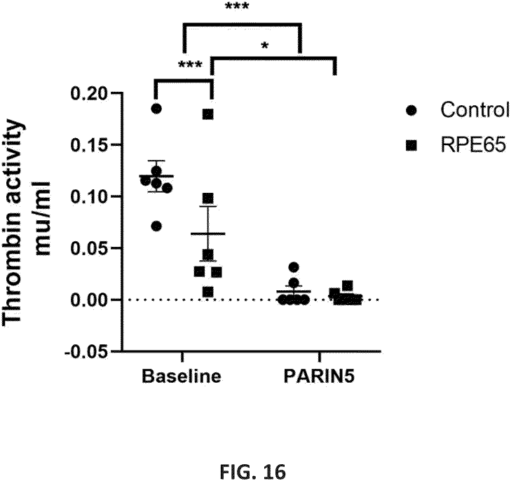

Indeed, the present inventors have demonstrated that treating STZ-induced diabetic mice with eye drops containing 100 nM PARIN5 for 5 weeks, protected the mouse retinal function, as shown in FIG. 6, and that treating neuroretina derived from a Retinitis pigmentosa (RP)-mouse model with 100 nM PARIN5 resulted in decreased thrombin activity, as shown in FIG. 16.

The present inventors have therefore uncovered and demonstrated that a potent treatment that protects retinal cells from degeneration can be performed simply by using ophthalmic formulation (e.g., eye drops) and administration, without the need to use gene therapy and/or invasive procedures that involve systemic administration and/or intervention. The use of molecules such as PARIN5 and similar molecules, as described, for example, in WO 2015/173802, is further advantageous by exhibiting no effect on blood coagulation, and as it does not block other anti-inflammatory downstream pathways induced by PAR1 activation (in comparison to PAR1 antagonist) and does not cause bleeding as direct thrombin inhibitors.

Embodiments of the present invention therefore relate to novel ophthalmic formulations and to easily accessible, affordable non-systemic and noninvasive treatment utilizing such a formulation, which protects the neuroretina from degeneration and can delay or prevent vision loss in subjects susceptible to such degeneration, thereby improving their quality of life and health. The novel ophthalmic formulation is usable in treating retinal disorders, particularly retinal disorders that are associated with or triggered by overexpression and/or activity of PAR1, and/or which are treatable by downregulating PAR1 activity and/or expression, including, but not limited to, retinal degeneration and associated diseases and disorders, as described herein in further detail, retinal dystrophy, retinal inflammation and retinal proliferative diseases and disorders such as retinal tumors.

### Ophthalmic Formulations

According to an aspect of some embodiments of the present invention there is provided an ophthalmic formulation.

By “ophthalmic formulation” it is meant a formulation that is suitable for topical application/administration (ophthalmic administration) to an eye of a subject.

The ophthalmic formulation of the present embodiments is designed based on the present findings according to which protease activator receptor 1 (PAR1) is present within the photoreceptors and inner retinal cells in the retinal tissue and is overexpressed and/or has increased activation in cases of retinal diseases and disorders such as, for example, retinal degeneration (e.g., observed in diabetic subjects), and according to which thrombin activity in a retinal tissue is elevated in such cases.

According to these findings, locally administering to an eye of a subject a formulation that comprises an active agent that can downregulate PAR1 activity and/or expression and/or interfere with its interaction with a protease such as thrombin, can protect the retinal tissue from degeneration.

Any PAR1 antagonist and/or protease inhibitor such as thrombin inhibitor and/or an agent that downregulates PAR1 activity and/or expression and/or interfere with its interaction with a protease such as thrombin can be included as an active agent in the formulation. Examples include, without limitation, T-L-C-K (also known as N alpha-tosyl-L-lysine chloromethyl ketone or TLCK), NAPAP (also known as Na-(2-naphthyl-sulphonyl-glycyl)-DL-p-amidinophenylalanyl-piperidine), PN-1 (also known as Protease nexin-1), PN-2 (also known as Protease nexin-2, APP) and SCH79797 (also known as N3..Cyclopropyl-7..[[4..(1.. methylethyl)phenyl]methyl]-7H-pyrrolo[3,2-f]quinazoline-1,3-diamine dihydrochloride), TLCK is an irreversible inhibitor of the serine protease trypsin (inactivates trypsin most rapidly at pH 7.5), and many trypsin-like serine proteases. The histidine-46 residue located in the active site of trypsin is alkylated by TLCK. NAPAP binds thrombin in the SI, S2 and S4 pockets. The amidine group on NAPAP forms a bidentate salt-bridge with Asp deep in the SI pocket, the piperidine group takes the role of proline residue and binds in the S2 pocket, and the naphthyl rings of the molecule forms a hydrophobic interaction with Trp in the S4 pocket. PN-1 is a 43 kDa thrombin inhibitor, member of the serine protease inhibitor superfamily (serpins), which regulates matrix accumulation and coagulation under pathophysiologic conditions by inhibiting thrombin, plasmin, and tissue plasminogen activators. PN-2 is a protease inhibitor, which is the secreted form of the amyloid beta-protein precursor (APP) which contains a Kunitz protease inhibitor domain. SCH79797 is a potent and selective non-peptide antagonist of protease activated receptor-1 (PAR1).

According to preferred embodiments of the present invention, the active agent that is included in the ophthalmic formulation is, alternatively or in addition to the exemplary agents listed above, a peptide conjugate such as described in WO 2015/173802, which is incorporated by reference as if fully set forth herein, including any of the embodiments described therein and any combination thereof.

According to some of any of the embodiments described herein, the agent is represented by Formula I:

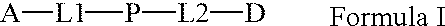

wherein:
P is a peptide moiety that comprises of at least 3 amino acid residues, as is further detailed hereinafter;
A is an N-terminus protecting group;
D is a moiety capable of interfering with a PAR1/protease interaction, which is also referred to herein as a protease-disabling moiety; and
L1 and L2 are each independently a linking moiety (linker) or absent.

Such an agent of Formula I is also referred to herein as a peptide conjugate, which comprises a peptide moiety as described herein, linked, directly or via a linker, at its N-terminus, to the A moiety or group, and conjugated to the D moiety at its C-terminus, directly or via a linker.

According to embodiments of the present invention, the peptide moiety P in Formula I comprises or consists of the amino acid sequence Asp-Pro-Arg (DPR).

According to exemplary embodiments, the peptide moiety P in Formula I is a three amino acid (3AA) moiety, which consists of the amino acid sequence Asp-Pro-Arg (DPR).

According to exemplary embodiments, the peptide moiety P in Formula I has 5 amino acid residues.

According to exemplary embodiments, the peptide moiety P in Formula I has an amino acid sequence as set forth in SEQ ID NO:2 (TLDPR).

The peptide moiety described herein is based on, or derived from, the thrombin binding site on PAR1, particularly, the binding site at the C-terminus of PAR1. In some embodiments, the peptide is derived from the sequence E³⁰SKATNATLDPR⁴¹ as set forth in SEQ ID NO:9.

In some embodiments, the peptide moiety P in Formula I comprises the amino-acid sequence DPR, LDPR (SEQ ID NO: 1), TLDPR (SEQ ID NO: 2), ATLDPR (SEQ ID NO: 3), NATLDPR (SEQ ID NO: 4), TNATLDPR (SEQ ID NO: 5), ATNATLDPR (SEQ ID NO: 6), KATNATLDPR (SEQ ID NO: 7), SKATNATLDPR (SEQ ID NO: 8), ESKATNATLDPR (SEQ ID NO: 9), PESKATNATLDPR (SEQ ID NO: 10), RPESKATNATLDPR (SEQ ID NO: 11), RRPES KATN ATLDPR (SEQ ID NO: 12), ARRPES KATNATLDPR (SEQ ID NO: 13), RARRPESKATNATLDPR (SEQ ID NO: 14), TRARRPESKATNATLDPR (SEQ ID NO: 15), RTRARRPESKATNATLDPR (SEQ ID NO: 16) and ARTRARRPESKATNATLDPR (SEQ ID NO: 17). Each possibility represents a separate embodiment of the present invention.

In some embodiments, the peptide moiety P in Formula I may consist of Asp-Pro-Arg (DPR). In some embodiments, the peptide moiety may consist of SEQ ID NO: 1. In some embodiments, the peptide moiety may consist of SEQ ID NO: 2. In some embodiments, the peptide moiety may consist of SEQ ID NO: 3. In some embodiments, the peptide moiety may consist of SEQ ID NO: 4. In some embodiments, the peptide moiety may consist of SEQ ID NO: 5. In some embodiments, the peptide moiety may consist of SEQ ID NO: 6. In some embodiments, the peptide moiety may consist of SEQ ID NO: 7. In some embodiments, the peptide moiety may consist of SEQ ID NO: 8. In some embodiments, the peptide moiety may consist of SEQ ID NO: 9. In some embodiments, the peptide moiety may consist of SEQ ID NO: 10. In some embodiments, the peptide moiety may consist of SEQ ID NO: 11. In some embodiments, the peptide moiety may consist of SEQ ID NO: 12. In some embodiments, the peptide moiety may consist of SEQ ID NO: 13. In some embodiments, the peptide moiety may consist of SEQ ID NO: 14. In some embodiments, the peptide moiety may consist of SEQ ID NO: 15. In some embodiments, the peptide moiety may consist of SEQ ID NO: 16. In some embodiments, the peptide moiety may consist of SEQ ID NO: 17.

In some embodiments, the peptide moiety may comprise an amino-acid sequence Asp-Pro-Arg or an active variant thereof. In some embodiments, the peptide moiety may comprise an amino-acid sequence set forth in SEQ ID NO: 1 or an active variant thereof. In some embodiments, the peptide moiety may comprise an amino-acid sequence set forth in SEQ ID NO: 2 or an active variant thereof. In some embodiments, the peptide moiety may comprise an amino-acid sequence set forth in SEQ ID NO: 3 or an active variant thereof. In some embodiments, the peptide moiety may comprise an amino-acid sequence set forth in SEQ ID NO: 4 or an active variant thereof. In some embodiments, the peptide moiety may comprise an amino-acid sequence set forth in SEQ ID NO: 5 or an active variant thereof. In some embodiments, the peptide moiety may comprise an amino-acid sequence set forth in SEQ ID NO: 6 or an active variant thereof. In some embodiments, the peptide moiety may comprise an amino-acid sequence set forth in SEQ ID NO: 7 or an active variant thereof. In some embodiments, the peptide moiety may comprise an amino-acid sequence set forth in SEQ ID NO: 8 or an active variant thereof. In some embodiments, the peptide moiety may comprise an amino-acid sequence set forth in SEQ ID NO: 9 or an active variant thereof. In some embodiments, the peptide moiety may comprise an amino-acid sequence set forth in SEQ ID NO: 10 or an active variant thereof. In some embodiments, the peptide moiety may comprise an amino-acid sequence set forth in SEQ ID NO: 11 or an active variant thereof. In some embodiments, the peptide moiety may comprise an amino-acid sequence set forth in SEQ ID NO: 12 or an active variant thereof. In some embodiments, the peptide moiety may comprise an amino-acid sequence set forth in SEQ ID NO: 13 or an active variant thereof. In some embodiments, the peptide moiety may comprise an amino-acid sequence set forth in SEQ ID NO: 14 or an active variant thereof. In some embodiments, the peptide moiety may comprise an amino-acid sequence set forth in SEQ ID NO: 15 or an active variant thereof. In some embodiments, the peptide moiety may comprise an amino-acid sequence set forth in SEQ ID NO: 16 or an active variant thereof. In some embodiments, the peptide moiety may comprise an amino-acid sequence set forth in SEQ ID NO: 17 or an active variant thereof.

The terms “active variant”, “analogue” and “variant” as used herein are interchangeable and refer to any peptide moiety that differs from a peptide sequence as set forth in DPR and any one of SEQ ID NO:] to SEQ ID NO: 17 by at least one amino-acid substitution, yet retains at least 70 %, optionally at least 80 % or at least 90 % or at least 95 %, or at least 98 %, or at least 99 % of the biological activity of the peptide moiety sequence from which it was derived, or to which it is most similar to. These terms also encompass peptides comprising regions having substantial similarity to the peptide moiety, such as structural variants.

The term “substantial similarity” means that two peptide sequences, when optimally aligned, share at least 50 percent sequence identity, at least 60 percent sequence identity, at least 70 percent sequence identity, at least 80 percent sequence identity, at least 90 percent sequence identity, or at least 95 percent sequence identity or more (e.g., 99 percent sequence identity). Typically, residue positions, which are not identical, differ by conservative amino acid substitutions.

In some embodiments, one or more of the peptide moieties may correspond to variants of the amino-acid sequence DPR or the amino acid sequences set forth in SEQ ID NO: 1 to SEQ ID NO: 17. Each possibility represents a separate embodiment of the present invention.

In some embodiments, the variants may comprise conservative substitutions relative to the amino acid sequence of the peptide moiety corresponding thereto.

Examples of conservative substitutions as considered in the present invention are the substitution of any positively-charged amino-acid (e.g., Arg, His, Lys) with any other positively-charged amino-acid; the substitution of any negatively-charged amino-acid (e.g., Asp, Glu) with any other negatively-charge amino-acid; the substitution of any polar-uncharged amino-acid (e.g., Ser, Thr, Asn, Gin) with any other polar-uncharged amino-acid; or the substitution of any hydrophobic amino-acid (e.g., Ala, Gly, Leu, Met, Phe, Trp, Tyr, Val) with any other hydrophobic amino-acid.

Thus, in some embodiments, an active variant may comprise Arg/His/Lys substitution; Asp/Glu substitution; Ser/Thr/Asn/Gln substitution; Ala/Ile/Leu/Met/Phe/Trp/Tyr/Val substitution; or any combination of the above. Each possibility represents a separate embodiment of the present invention.

In some embodiments, the peptide may be selected from the amino-acid sequence DPR and those set forth in SEQ ID NOs: 1 to 17, wherein at least on proline is substituted with a positive-charge amino acid. In other embodiments, the peptide is selected from DPR and SEQ ID NOs: 1 to 17, wherein at least one proline is substituted with lysine. Without being bound by any theory of mechanism, the peptide is substituted in order to obtain improved specificity to thrombin and potentially other coagulation factors, improved penetration into posterior segment and retina cells and prolonged half-life of the conjugate.

Residue positions, which are not identical, may also be composed of peptide analogs, including unnatural amino acids or derivatives of such. Analogs typically differ from naturally occurring peptides at one, two or a few positions, often by virtue of conservative substitutions. The substituting positive-charged, negative charged, polar, hydrophobic, etc. amino acid residues can be selected from naturally-occurring and non-naturally occurring amino acids, as described hereinafter.

Some analogs may also include non-naturally occurring amino acids or modifications of N- or C- terminal amino acids at one, two or a few positions. Examples of non-naturally occurring amino acids, without limiting to, are D-amino acids, alpha, alpha-disubstituted amino acids, N-alkyl amino acids, lactic acid, 4-hydroxyproline, y-carboxyglutamate, epsilon-N,N,N-tri methyllysine, epsilon-N-acetyllysine, O-phosphoserine, N-acetylserine, N-formylmethionine, 3-methylhistidine, 5-hydroxylysine, omega-N-methylarginine, and isoaspartic acid.

Active variants may also include peptide moieties that feature one or more modification as the peptide bond linking two adjacent amino acid residues, as described hereinafter.

The term “peptide” as used herein encompasses native peptides (either degradation products, synthetically synthesized peptides or recombinant peptides) and peptidomimetics (typically, synthetically synthesized peptides), as well as peptoids and semipeptoids which are peptide analogs, which may have, for example, modifications rendering the peptides more stable while in a body or more capable of penetrating into cells. Such modifications include, but are not limited to, N-terminus modification, C-terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C.A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylene bonds (—CO—CH₂—), α-aza bonds (—NH—N(R)—CO—), wherein R is any alkyl, e.g., methyl, carba bonds (—CH₂—NH—), hydroxyethylene bonds (—CH(OH)—CH₂—), thioamide bonds (—CS—NH—), olefinic double bonds (—CH═CH—), retro amide bonds (—NH—CO—), peptide derivatives (-N(R)—CH₂—CO—), wherein R is the “normal” side chain, naturally presented on the carbon atom. These modifications can occur at any of the bonds along the peptide chain and even at several (2-3) at the same time.

As used herein throughout, the term “amino acid” or “amino acids” is understood to include the 20 naturally occurring amino acids; those amino acids are often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Tables A and B below list naturally occurring amino acids (Table A) and non-conventional or modified amino acids (Table B) which can be used with the present invention.

TABLE A

| | | |
|---|---|---|
| Amino Acid | Three-Letter Abbreviation | One-letter Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic Acid | Glu | E |
| Glycine | Gly | G |
| Histidine | His | H |
| isoleucine | Iie | I |
| Leucine | Leu | L |
| Lysine | Lys | K |
| Methionine | Met | M |
| phenylalanine | Phe | F |
| Proline | Pro | P |
| Serine | Ser | S |
| Threonine | Thr | T |
| tryptophan | Trp | W |
| tyrosine | Tyr | Y |
| Valine | Val | V |
| Any amino acid as above | Xaa | X |

TABLE B

| | | | |
|---|---|---|---|
| Non-conventional amino acid | Code | Non-conventional amino acid | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgin |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcyclopentylalanine | Mcpen |
| D-α-methylasparagine | Dmasn | α-methyl-α-napthylalanine | Manap |
| D-α-methylaspartate | Dmasp | α- methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N- amino-a-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cyclododeclglycine | Ncdod |
| D-α-methylalnine | Dnmala | N-cyclooctylglycine | Ncoct |
| D-α-methylarginine | Dnmarg | N-cyclopropylglycine | Ncpro |
| D-α-methylasparagine | Dnmasn | N-cycloundecylglycine | Ncund |
| D-α-methylasparatate | Dnmasp | N-(2,2-diphenylethyl)glycine | Nbhm |
| D-α-methylcysteine | Dnmcys | N-(3,3-diphenylpropyl)glycine | Nbhe |
| D-N-methylleucine | Dnmleu | N-(3-indolylyethyl) glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nva |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomo phenylalanine | Mhphe |
| L-α-methylisoleucine | Mile | N-(2-methylthioethyl)glycine | Nmet |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl)glycine | Nser |
| D-N-methylisoleucine | Dnmile | N-(imidazolylethyl)glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | Penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | mser | L-α-methylthreonine | Mthr |
| L-α-methylvaline | Mtrp | L-α-methyltyrosine | Mtyr |
| L-α-methylleucine | Mval Nnbhm | L-N-methylhomophenylalanine | Nmhphe |
| N-(N-(2,2-diphenylethyl) | | N-(N-(3,3-diphenylpropyl) | |
| carbamylmethyl-glycine | Nnbhm | carbamylmethyl(1)glycine | Nnbhe |
| 1-carboxy-1-(2,2-diphenyl ethylamino)cyclopropane | Nmbc | | |

(Table B; Cont.)

The moiety A in Formula I is an amine-protecting group, preferably a protecting group usable as an alpha-amine protecting group in peptide chemistry.

The terms “protecting moiety” and “PRO” are interchangeable and are used herein in the context of moiety A in Formula I, and refer to any moiety capable of protecting the peptide conjugate of the present embodiments from adverse effects such as proteolysis, degradation or clearance, or alleviating such adverse effects. Alternatively, or is addition, the protecting moiety may function as an end-capping moiety, which “masks” the positive charge of the peptide conjugate at physiological pH. This moiety is therefore also referred to herein as “N.-terminus end-capping moiety”.

In some embodiments, the protecting moiety may be an alpha-amino protecting moiety. In some embodiments, the protecting moiety may be tosyl (a tosyl group) or derivatives thereof. In some embodiments, the alpha-amino protecting moiety may be tosyl.

The phrase “end-capping moiety”, as used herein, refers to a moiety that when attached to the terminus of the peptide, modifies the end-capping. The end-capping modification typically results in masking the charge of the peptide terminus, and/or altering chemical features thereof, such as, hydrophobicity, hydrophilicity, reactivity, solubility and the like. Examples of moieties suitable for peptide end-capping modification can be found, for example, in Green et al., “Protective Groups in Organic Chemistry”, (Wiley, 2^{nd} ed. 1991) and Harrison et al., “Compendium of Synthetic Organic Methods”, Vols. 1-8 (John Wiley and Sons, 1971-1996).

Representative examples of N-terminus end-capping moieties include, but are not limited to, formyl, acetyl (also denoted herein as “Ac”), trifluoroacetyl, benzyl, benzyloxycarbonyl (also denoted herein as “Cbz”), tert-butoxycarbonyl (also denoted herein as “Boc”), trimethylsilyl (also denoted “TMS”), 2-trimethylsilyl-ethanesulfonyl (also denoted “SES”), trityl and substituted trityl groups, allyloxycarbonyl, 9-fluorenylmethyloxycarbonyl (also denoted herein as “Fmoc”), and nitro-veratryloxycarbonyl (“NVOC”).

In some embodiments the protecting moiety A in Formula I is selected from t-butyloxycarbonyl, t-amyloxycarbonyl, adamantyl-oxycarbonyl, p-methoxybenzyloxycarbonyl, 9-fluorenylmethoxycarbonyl (FMOC), 2-chlorobenzyloxycarbonyl, tosyl (CH₃C₆H₄SO₂—), benzyloxycarbonyl (CBz), adamantyloxycarbonyl, 2,2,5,7, 8-pentamethylchroman-6-sulfonyl, 2,3,6-trimethyl-4-methoxyphenylsulfonyl, t-butyl benzyl (BZl) or substituted BZl, 1 p-methoxybenzyl, p-nitrobenzyl, p-chlorobenzyl, o-chlorobenzyl, and 2,6-dichlorobenzyl. Each possibility represents a separate embodiment of the present invention.

In some embodiments, PRO may be selected from t-butyl, cyclohexyl, cyclopentyl, benzyloxymethyl (BOM), tetrahydropyranyl, trityl, chlorobenzyl, 4-bromobenzyl, and 2,6-dichlorobenzyl. Each possibility represents a separate embodiment of the present invention.

Other protecting groups which may suitably employed are bromobenzyloxycarbonyl, xanthyl (Xan) and p-methoxybenzyl. Each possibility represents a separate embodiment of the present invention.

In some embodiments, the A moiety is or comprises tosyl, or a tosyl derivative in which the toluyl group is further substituted by one or more substituents (e.g., one or more alkyl, cycloalkyl, benzyl, aryl, etc.).

In exemplary embodiments, A in Formula I is an aromatic N-terminus protecting moiety or group. Examples of such moieties include, without limitation, benzyl, benzyloxymethyl (130 M), tetrahydropyranyl, trityl, chlorobenzyl, 4-bromobenzyl, and 2,6-dichlorobenzyl, bromobenzyloxycarbonyl, xanthyl (Xan), p-methoxybenzyl, methoxybenzyloxycarbonyl, 9-fluorenylmethoxycarbonyl (FMOC), 2-chlorobenzyloxycarbonyl, tosyl (CH₃C₆H₄SO₂—), benzyloxycarbonyl (CBz), 2,3,6-trimethyl-4-methoxyphenylsulfonyl, t-butyl benzyl (BZl), p-nitrobenzyl, p-chlorobenzyl, o-chlorobenzyl, and 2,6-dichlorobenzyl.

The moiety D in Formula I is capable of interfering with a protease activity and/or with an interaction between a protease and PAR1, and thus with an activation of the protease. This moiety is also referred to herein as a “protease-disabling moiety” or “DIS” and encompasses, for example, any moiety that is capable of binding to a protease and transiently or permanently disable its proteolytic activity. In some embodiments, the protease-disabling moiety may be a thrombin-disabling moiety. In some embodiments, the protease disabling moiety may be a thrombin inhibitor.

In some embodiments, the protease-disabling moiety may be a protease-disabling compound selected from irreversible inhibitors and reversible inhibitors of the respective protease.

In some embodiments, the protease-disabling moiety may be an irreversible inhibitor such as, but not limited to, optionally substituted acetyl such as, for example, chloromethylketone (—C(═O)CH₂Cl), a sulfonylfluoride (—SO₂F), a carboxylate (—C(═O)OR), a borate (—B(OR)₂) and combinations thereof.

In some embodiments, the protease-disabling moiety may be a reversible inhibitor such as, but not limited to, an aldehyde (—CHO), an arylketone (—C(═O)—Aryl), trifluoromethylketone (—C(═O)CF₃) a ketocarboxylate (—C(═O)C(═O)OR) and combinations thereof.

R is as defined hereinbelow.

In exemplary embodiments the protease-disabling moiety is selected from chloromethylketone (CK or CMK) and derivatives thereof, a sulfonylfluoride (—SO₂F), a carboxylate, a borate, an aldehyde, an aryl ketone, a trifluoromethylketone and a ketocarboxylic acid.

In exemplary embodiments, the protease-disabling moiety is or comprises a substituted acetyl. In exemplary embodiments, the substituted acetyl may be a haloacetyl. In exemplary embodiments, the haloacetyl is a chloroacetyl. In exemplary embodiments, the protease-disabling moiety is chloromethylketone (CK or CMK). Alternatively, the protease-disabling moiety is bromomethylketone, iodomethylketone, chloroethylketone, bromoethylketone, iodoethylketone, chloropropylketone, bromoprpylketone, iodopropylketone, and other haloalkylketones, where the alkyl is of 1 to 8, or from 1 to 6, carbon atoms in length.

In exemplary embodiments, the peptide conjugate of Formula I is tosyl-DPR-CMK. In exemplary embodiments, the peptide conjugate of Formula I is tosyl-TLDPR-CK.

In exemplary embodiments, the peptide conjugate of Formula I is tosyl-P-CMK, wherein P is a peptide moiety as defined herein in any of the respective embodiments and any combination thereof.

The linking moiety or linker, L1 and/or L2, can be, for example, selected from amino-acid moieties, peptide moieties, nucleotide moieties, oligonucleotide moieties etc. Contemplated linkers may also serve a further therapeutic purpose, for example, they may be fluorescent, thereby enabling detection of the peptide conjugates carrying them, or they may be a polyethylene glycol (PEG) moiety, further protecting the peptide conjugates carrying them from degradation. Each possibility represents a separate embodiment of the present invention.

The linking moiety or linker, L1 and/or L2, can alternatively be, for example, a hydrocarbon chain, as defined herein, or an alkylene glycol, as defined herein.

In some of any of the embodiments described herein, L1 is absent, such that the N-terminus protecting moiety is directly attached to the terminal amino acid of the peptide moiety P, through the alpha amine or through its side chain.

In some of any of the embodiments described herein, L2 is absent, such that the D moiety is linked to the C-terminus of the peptide moiety or to the C terminal amine acid of the peptide moiety via its side chain, directly.

An ophthalmic formulation as described herein further comprises a pharmaceutically acceptable carrier, preferably, an ophthalmically acceptable carrier.

As used herein, the term “pharmaceutically acceptable carrier” describes a carrier or a diluent that is used to facilitate the administration of the active agent and which does not cause significant irritation to an organism and does not abrogate the biological activity and properties of the administered active materials. Examples, without limitations, of carriers include water, buffered aqueous solutions, propylene glycol, emulsions and mixtures of organic solvents with water, as well as solid (e.g. powdered or polymeric or particulated) and gaseous carriers.

Techniques for formulation and administration of drugs may be found in “Remington’s Pharmaceutical Sciences” Mack Publishing Co., Easton, PA, latest edition, which is incorporated herein by reference.

Compositions for use in accordance with the present embodiments thus may be formulated in conventional manner using one or more pharmaceutically acceptable carriers, excipients and/or auxiliaries, which facilitate processing of the compounds into preparations which can be used pharmaceutically. The dosage may vary depending upon the dosage form employed.

The pharmaceutically acceptable carrier can be either an organic carrier or an aqueous carrier. In some embodiments, the carrier is an aqueous carrier. An aqueous carrier is preferably an ophthalmically acceptable carrier, for example, which comprises or is purified water, such as, for example, distilled and deionized water.

Aqueous formulations are preferred since these formulations are suitable for ophthalmic administration. However, non-aqueous formulations are also contemplated. For example, in cases where the formulation is in a form of a paste or an emulsion, non-aqueous carriers or mixed carriers of aqueous and organic carriers can be used.

According to some embodiments, the formulation is formulated for topical, ophthalmic application, as a topical dosage form.

As used herein, the phrase “topical dosage form” describes a dosage form suitable for topical administration to the eye.

The topical dosage form described herein can be, for example, in a form of a powder, granules, a cream, an ointment, a paste, a gel, a lotion, a milk, a suspension, an aerosol, a spray, a foam, a gauze, a wipe, a sponge, a wound dressing, a pledget, a patch, a pad, an adhesive bandage, and a non-adhesive bandage.

Depending on the selected carrier, the formulation can be in a form of a liquid (e.g., a solution), an emulsion, a gel, an aerosol, a spray, a foam, a mousse, an ointment, a paste, a lotion, or a suspension.

In some embodiments, the formulation is formulated as a liquid reservoir, to be applied as drops, spray, aerosol, liquid, foam and the like. Suitable carriers and other ingredients are used in these cases. For example, for application as an aerosol or foam, a propellant is used. For application as foam, foam-forming agents can also be used.

In some embodiments, the formulation is in a form of a cream. Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. An exemplary cream formulation can be obtained by mixing the active agent described herein with a carrier comprising cellulose derivatives such as cellulose acetate, hydroxyethyl cellulose and/or a polyethylene glycol.

In some embodiments, the formulation is a form of an ointment. Ointments are semisolid preparations, typically based on petrolatum or petroleum derivatives. The specific ointment base to be used is one that provides for optimum delivery for the active agent chosen for a given formulation. As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

In some embodiments, the formulation is in a form of a paste. Pastes are semisolid dosage forms in which the active agent is suspended in a suitable base. Depending on the nature of the base, pastes are divided between fatty pastes or those made from a single-phase aqueous gels. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

In some embodiments, the formulation is in a form of a gel. Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

In some embodiments, the formulation is in a form of a foam. Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or hydroalcoholic, but are typically formulated with high alcohol content which, upon application to the treated area, quickly evaporates, driving the composite material to the site of treatment.

In some embodiments, a topical dosage form includes a solid or semi-solid substrate, e.g., a gauze, a wipe, a bandage, a pad, a pledget, a sponge, a mesh, a fabric, and the likes, and the formulation is incorporated in and/or on the substrate.

The substrate in such topical dosage forms can be of any form and materials used to make up gauzes, wipes, bandages, pads, pledgets, sponges, meshes, fabrics (woven and non-woven, cotton fabrics, and the like), and any other substrates commonly used in medical applications.

Such topical dosage forms may optionally further comprise an adhesive, for example, for facilitating the topical application of the formulation to the eye for a prolonged time period.

Exemplary adhesives include, but are not limited to, medically acceptable bioadhesives, polymer glues, etc., and can be applied to the substrate by, for example, dip coating with an adhesive base. Such dip coating can be effected during manufacture of the substrate, or at any time prior to its application. In some embodiments, the composite material can be embedded within and/or on the material of the substrate, for example, embedded into or onto a polymer or fabrics by application of heat, or fused to the substrate. In other embodiments, the composite material can be incorporated into the base material of the substrate, for example, mixed within the components of a polymer before polymerization, or mixed with components forming fibers used to make up a gauze or a mesh or pad, etc.

In exemplary embodiments, the formulation is a liquid formulation, which can be, for example, in a form of a solution, an emulsion, or a suspension, of the active agent as described herein and a liquid carrier.

In exemplary embodiments, the carrier is water or an aqueous solution in which the active agent is dissolved, suspended or dispersed.

In some of any of the embodiments described herein the formulation is such that features a pH that ranges from about 3.0 to about 8.0, or from about 3.5 to about 8.0, or from about 3.5 to about 7.5 or to about 7.4, or from about 4.0 to about 7.5, or from about 4.5 to about 7.5, or from about 3.5 to about 6.5, or from about 3.5 to about 6.0, or from about 4.0 to about 8.0, or from about 6.0 to about 8.0, or from about 6.0 to about 7.5, or from about 6.5 to about 7.5, or from about 7.0 to about 7.4, or is about a physiological pH (e.g. of the retina).

In exemplary embodiments, the carrier is or comprises a buffer, and can be, for example, a phosphate-buffered saline carrier (PBS).

Exemplary buffers generally include borates, borate-polyol complexes, succinate, phosphate buffering agents, citrate buffering agents, acetate buffering agents, carbonate buffering agents, organic buffering agents, amino acid buffering agents, or combinations thereof.

Exemplary borates include boric acid, salts of boric acid, other pharmaceutically acceptable borates, and combinations thereof. In some cases, borates include boric acid, sodium borate, potassium borate, calcium borate, magnesium borate, manganese borate, and other such borate salts.

Exemplary polyols include any compound having at least one hydroxyl group on each of two adjacent carbon atoms that are not in trans configuration relative to each other. In some embodiments, the polyols is linear or cyclic, substituted or unsubstituted, or mixtures thereof, so long as the resultant complex is water soluble and pharmaceutically acceptable. Non-limiting examples of polyols include sugars, sugar alcohols, sugar acids and uronic acids, for example, but are not limited to: mannitol, glycerin, xylitol and sorbitol.

Exemplary phosphate buffering agents include, without limitation, phosphoric acid; alkali metal phosphates such as disodium hydrogen phosphate, sodium dihydrogen phosphate, trisodium phosphate, dipotassium hydrogen phosphate, potassium dihydrogen phosphate, and tripotassium phosphate; alkaline earth metal phosphates such as calcium phosphate, calcium hydrogen phosphate, calcium dihydrogen phosphate, monomagnesium phosphate, dimagnesium phosphate (magnesium hydrogen phosphate), and trimagnesium phosphate; ammonium phosphates such as diammonium hydrogen phosphate and ammonium dihydrogen phosphate; or a combination thereof. In some instances, the phosphate buffering agent is an anhydride. In some instances, the phosphate buffering agent is a hydrate.

Exemplary borate-polyol complexes include those described in U.S. Pat. No. 6,503,497.

Exemplary citrate buffering agents include citric acid and sodium citrate.

Exemplary acetate buffering agents include acetic acid, potassium acetate, and sodium acetate.

Exemplary carbonate buffering agents include sodium bicarbonate and sodium carbonate.

Exemplary organic buffering agents include Good’s Buffer, such as for example 2-(N-morpholino)ethanesulfonic acid (MES), N-(2-Acetamido)iminodiacetic acid, N-(Carbamoylmethyl)iminodiacetic acid (ADA), piperazine-N,N′-bis(2-ethanesulfonic acid (PIPES), N-(2-acetamido)-2-aminoethanesulfonic acid (ACES), beta.-Hydroxy-4-morpholinepropanesulfonic acid, 3-Morpholino-2-hydroxypropanesulfonic acid (MOPSO), cholamine chloride, 3-(N-morpholino)propansulfonic acid (MOPS), N,N-bis(2-hydroxyethyl)-2-aminoethanesulfonic acid (BES), 2-[(2-Hydroxy-1,1-bis(hydroxymethyl)ethyl)amino]ethanesulfonic acid (TES), 4-(2-hydroxyethyl)-1-piperazineethanesulfonic acid (HEPES), 3-(N,N-Bis[2-hydroxyethyl]amino)-2-hydroxypropanesulfonic acid (DIPSO), acetamidoglycine, 3-{[1,3-Dihydroxy-2-(hydroxymethyl)-2-propanyl]amino}-2-hydroxy-1-propane- ssulfonic acid (TAPSO), piperazine-1,4,-bis (2-hydroxypropanesulphonic acid) (POPSO), 4-(2-hydroxyethyl)piperazine-1-(2-hydroxypropanesulfonic acid) hydrate (HEPPSO), 3-[4-(2-hydroxyethyl)-1-piperazinyl]propanesulfonic acid (HEPPS), tricine, glycinamide, bicine or N-tris(hydroxymethyl)methyl-3-aminopropanesulfonic acid sodium (TAPS); glycine; and diethanolamine (DEA).

Exemplary amino acid buffering agents include taurine, aspartic acid and its salts (e.g., potassium salts, etc.), E-aminocaproic acid, and the like.

In some of any of the embodiments described herein, the ophthalmic formulation is an ophthalmic gel, and the ophthalmically acceptable carrier comprises water and at least one viscosity-enhancing agent. In some embodiments, the viscosity-enhancing agent is selected from cellulose-based polymers, polyoxyethylene-polyoxypropylene triblock copolymers, dextran-based polymers, polyvinyl alcohol, dextrin, polyvinylpyrrolidone, polyalkylene glycols, chitosan, collagen, gelatin, hyaluronic acid, or combinations thereof.

Exemplary ophthalmically acceptable viscosity agents include, but are not limited to, hydroxypropyl methylcellulose, hydroxy ethyl cellulose, polyvinylpyrrolidone, carboxymethyl cellulose, polyvinyl alcohol, sodium chondroitin sulfate, sodium hyaluronate. Other viscosity enhancing agents compatible with the targeted ocular site include, but are not limited to, acacia (gum arabic), agar, aluminum magnesium silicate, sodium alginate, sodium stearate, bladderwrack, bentonite, carbomer, carrageenan, Carbopol, xanthan, cellulose, microcrystalline cellulose (MCC), ceratonia, chitin, carboxymethylated chitosan, chondrus, dextrose, furcellaran, gelatin, Ghatti gum, guar gum, hectorite, lactose, sucrose, maltodextrin, mannitol, sorbitol, honey, maize starch, wheat starch, rice starch, potato starch, gelatin, sterculia gum, xanthum gum, gum tragacanth, ethyl cellulose, ethykdroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate,), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVT: povidone), Splenda® (dextrose, maltodextrin and sucralose) or combinations thereof. In some embodiments, the viscosity-enhancing excipient is a combination of MCC and CMC.

In some embodiments, the viscosity-enhancing agent is a combination of carboxymethylated chitosan, or chitin, and alginate. The combination of chitin and alginate with the ophthalmic agents disclosed herein acts as a controlled release formulation, restricting the diffusion of the ophthalmic agents from the formulation. Moreover, the combination of carboxymethylated chitosan and alginate is optionally used to assist in increasing the permeability of the ophthalmic agents in the eye.

In some embodiments, the formulation further comprises a gelling agent such as, but not limited to, one or more of celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum and any combinations or mixtures thereof. Other gelling agents include, but is not limited to, poloxamer (e.g., Poloxamer 407), tetronics, ethyl (hydroxyethyl) cellulose, cellulose acetate phthalate (CAP), carbopol (e.g. Carbopol 1342P NF, Carbopol 980 NF), alginates (e.g. low acetyl gellan gum (Gelrite®)), gellan, hyaluronic acid, pluronics (e.g., Pluronic F-127), chitosan, polyvinyl alcohol (PVA), polyvinylpyrrolidone (PVP), dextran, hydroxy propyl methyl cellulose (HPMC), hydroxyethylcellulose (HEC), methylcellulose (MC), thiolated xyloglucan, polymethacrylic acid (PMMA), polyethylene glycol (PEG), pseudolatexes, xyloglucans, or combinations thereof.

In some of any of the embodiments described herein, the ophthalmic formulation is an ophthalmic gel, and the ophthalmically acceptable carrier is a homogeneous, viscous, semi-solid preparation, most commonly a greasy, thick oil (e.g. oil 80%-water 20%) with a high viscosity. Exemplary types of ointment bases are: hydrocarbon bases, e.g. hard paraffin, soft paraffin, microcrystalline wax and ceresine; absorption bases, e.g. wool fat, beeswax; water soluble bases, e.g. macrogols 200, 300, 400; emulsifying bases, e.g. emulsifying wax, cetrimide; vegetable oils, e.g. olive oil, coconut oil, sesame oil, almond oil and peanut oil. Additional exemplary ointment bases include ophthalmically acceptable oil and fat bases, such as natural wax e.g. white and yellow bees wax, carnauba wax, wool wax (wool fat), purified lanolin, anhydrous lanolin, petroleum wax e.g. hard paraffin, microcrystalline wax; hydrocarbons e.g. liquid paraffin, white and yellow soft paraffin, white petrolatum, yellow petrolatum; or combinations thereof.

In exemplary embodiments, poly(ethylene-glycols), polyethoxylated castor oils, alcohols having 12 to 20 carbon atoms or a mixture of two or more of said components are effective excipients for dispersing and/or dissolving effective amounts of active agents, in an ointment base, in particular in an ointment base substantially comprising oleaginous and hydrocarbon components, and that the resulting ointments are tolerated by the skin and ocular tissue.

Ointments may include dispersing agents. Exemplary dispersing agents include, but are not limited to, a poly(ethylene-glycol), a polyethoxylated castor oil, an alcohol having 12 to 20 carbon atoms and a mixture of two or more of said components. Alcohols having 12 to 20 carbon atoms include particularly stearyl alcohol, cetyl alcohol and mixtures thereof.

In some of any of the embodiments described herein, the ophthalmic formulation comprises solid components, and may be in a form of nanoparticles or microparticles, optionally dispersed in a liquid or gel carrier as described herein. The ophthalmic formulation may, for example, comprise liposomes, niosomes, discosomes and/or dendrimers, in which the active agent is encapsulated. In some embodiments, the encapsulating matrix is biodegradable and in some embodiments, it is biodegradable upon being administered to eye, when exposed to the respective physiological conditions.

In some of any of the embodiments described herein, the ophthalmic formulation may further comprise one or more additional ingredients, which are aimed at improving or facilitating its preparation, application and/or performance. Such additional ingredients include, for example, anti-irritants, anti-foaming agents, humectants, deodorants, antiperspirants, preservatives, emulsifiers, occlusive agents, emollients, thickeners, penetration enhancers, colorants, propellants and/or surfactants, depending on the final form of the formulation.

Representative examples of humectants that are usable in this context of the present embodiments include, without limitation, guanidine, glycolic acid and glycolate salts (e.g. ammonium slat and quaternary alkyl ammonium salt), aloe vera in any of its variety of forms (e.g., aloe vera gel), allantoin, urazole, polyhydroxy alcohols such as sorbitol, glycerol, hexanetriol, propylene glycol, butylene glycol, hexylene glycol and the like, polyethylene glycols, sugars and starches, sugar and starch derivatives (e.g., alkoxylated glucose), hyaluronic acid, lactamide monoethanolamine, acetamide monoethanolamine and any combination thereof.

Suitable emulsifiers that can be used in the context of the present embodiments include, for example, one or more sorbitans, alkoxylated fatty alcohols, alkylpolyglycosides, soaps, alkyl sulfates, or any combinations thereof.

Suitable occlusive agents that can be used in the context of the present embodiments include, for example, petrolatum, mineral oil, beeswax, silicone oil, lanolin and oil-soluble lanolin derivatives, saturated and unsaturated fatty alcohols such as behenyl alcohol, hydrocarbons such as squalane, and various animal and vegetable oils such as almond oil, peanut oil, wheat germ oil, linseed oil, jojoba oil, oil of apricot pits, walnuts, palm nuts, pistachio nuts, sesame seeds, rapeseed, cade oil, corn oil, peach pit oil, poppyseed oil, pine oil, castor oil, soybean oil, avocado oil, safflower oil, coconut oil, hazelnut oil, olive oil, grape seed oil and sunflower seed oil.

Suitable emollients, that can be used in the context of the present embodiments include, for example, dodecane, squalane, cholesterol, isohexadecane, isononyl isononanoate, PPG ethers, petrolatum, lanolin, safflower oil, castor oil, coconut oil, cottonseed oil, palm kernel oil, palm oil, peanut oil, soybean oil, polyol carboxylic acid esters, derivatives thereof and mixtures thereof.

Suitable thickeners (viscosity enhancing agents) that can be used in the context of the present embodiments include, for example, non-ionic water-soluble polymers such as hydroxyethylcellulose (commercially available under the Trademark Natrosol® 250 or 350), cationic water-soluble polymers such as Polyquat 37 (commercially available under the Trademark Synthalen® CN), fatty alcohols, and mixtures thereof.

Additional suitable viscosity-enhancing agents include, but are not limited to, a cellulose-based polymer selected from cellulose gum, alkylcellulose, hydroxyl-alkyl cellulose, hydroxyl-alkyl alkylcellulose, carboxy-alkyl cellulose, or combinations thereof.

Suitable penetration/permeation enhancers usable in context of the present embodiments include, but are not limited to, polyethylene glycol monolaurate (PEGML), propylene glycol (PG), propylene glycol monolaurate (PGML), glycerol monolaurate (GML), lecithin, the 1-substituted azacycloheptan-2-ones, particularly 1-n-dodecylcyclazacycloheptan-2-one (available under the trademark Azone® from Whitby Research Incorporated, Richmond, Va.), alcohols, menthol, TWEENS such as TWEEN 20, and the like. The permeation enhancer may also be a vegetable oil. Such oils include, for example, safflower oil, cottonseed oil and corn oil.

Additional suitable permeation enhancers include, but are not limited to, surfactants (e.g. non-ionic surfactants), benzalkonium chloride, EDTA, surface-active heteroglycosides, calcium chelators, hydroxyl propyl beta cyclodextrin (HP beta CD), bile salts, and the like.

Penetration enhancers are materials that transiently increase the permeability of the corneal epithelium or conjunctiva to facilitate API penetration therethrough. The use of known percutaneous penetration enhancers in pharmaceutical compositions for ophthalmic administration has been proposed (see Sasaki et al. Crit. Rev. Ther. Drug Carrier Syst. 1999, 16, 85-146 and PCT patent publication WO 2006/082588).

Exemplary penetration enhancers include saponin and saponin derivatives, benzalkonium chloride, BL-9, deoxycholic acid, digitonin, escin, fusidic acid, fusidate, fusidic acid derivatives, sodium deoxycholate, acetone, acyl lactylates, acyl peptides, acylsarcosinates, alcohols, alkanolamine salts of fatty acids, alkyl benzene sulphonates, alkyl ether sulphates, alkyl sulphates, allantoin, anionic surface-active agents, 1-substituted azacycloheptan-2-ones, benzyl benzoate, benzyl salicylate, butan-1,4-diol, butyl benzoate, butyl laurate, butyl myristate, butyl stearate, cationic surface-active agents, citric acid, cocoamidopropylbetaine, decyl methyl sulfoxide, decyl oleate, dibutyl azelate, dibutyl phthalate, dibenzyl sebacate, dibutyl sebacate, dibutyl suberate, dibutyl succinate, dicapryl adipate, didecyl phthalate, diethylene glycol, diethyl sebacate, diethyl-m-toluamide, di(2-hydroxypropyl) ether, diisopropyl adipate, diisopropyl sebacate, N,N-dimethyl acetamide, dimethyl azelate, N,N-dimethyl formamide, 1,5-dimethyl-2-pyrrolidone, dimethyl sebacate, dioctyl adipate, dioctyl azelate, dioctyl sebacate, 1,4 dioxane, 1-dodecylazacyloheptan-2-one, dodecyl dimethyl amine oxides, ethyl caprate, ethyl caproate, ethyl caprylate, 2-ethyl-hexyl pelargonate, ethyl-2-hydroxypropanoate, ethyl laurate, ethyl myristate, 1-ethyl-2-pyrrolidone, ethyl salicylate, glycerol monolaurate, hexyl laurate, 2-hydroxyoctanoic acid, 2-hydroxypropanoic acid, 2-hydroxypropionic acid, isethionates, isopropyl isostearate, isopropyl palmitate, guar hydroxypropyltrimonium chloride, hexan-2,5-diol, khellin, lamepons, lauryl alcohol, lecithin, maypons, metal salts of fatty acids, methyl nicotinate, 2-methyl propan-2-ol, 1-methyl-2-pyrrolidone, 5-methyl-2-pyrrolidone, methyl taurides, miranol, nonionic surface-active agents, octyl alcohol, octylphenoxy polyethoxyethanol, oleic ethanolamide, pleyl alcohol, pentan-2,4-diol, phenoxyethanol, phosphatidyl choline, phosphine oxides, polyalkoxylated ether glycollates, poly(diallylpiperidinium chloride), poly(dipropyldiallylammonium chloride), polyethylene glycol monolaurate, polyglycerol esters, poly(vinyl pyridinium chloride), propan-1-ol, propan-2-ol, propylene glycol, propylene glycol dipelargonate, propylene glycol monolaurate, pyroglutamic acids, 2-pyrrolidone, pyruvic acids, Quaternium 5, Quaternium 18, Quaternium 19, Quaternium 23, Quaternium 31, Quaternium 40, Quaternium 57, quartenary amine salts, quaternised poly (dimethylaminoethylmethacrylate), quaternised poly (vinyl alcohol), sapamin hydrochloride, sodium cocaminopropionate, sodium dioctyl sulphonsuccinate, sodium laurate, sodium lauryl ether sulphate, sodium lauryl sulphate, sorbitan monooleate, sorbitan monolaurate, sugar esters, sulphosuccinate, tetrahydrofuran, tetrahydrofurfural alcohol, transcutol, triethanolamine dodecyl benzene sulphonate, triethanolamine oleate, urazole, urea, ammonium glycyrrhizide, Brij 35, Brij 78, Brij-98, cetylpyridium chloride, chenodeoxycholic acid, cholate, cholic acid, decamethonium, decamethonium bromide, dimethyl sulphoxide, EDTA and disodium EDTA, glycocholate, glycocholic acid, glycodeoxycholic acid, glycyrrhizic acid, paraben, polyoxyethylene, polyoxyethylene ethers of fatty acids such as polyoxyethylene 4-, 9-, 10-, and 23-lauryl ether, polyoxyethylene 10- and 20-cetyl ether, polyoxyethylene 10- and 20-stearyl ether, polyoxyethylated castor oil, polyoxyethylene monolaurate, polyoxyethylene sorbitans such as polyoxyethylene sorbitan monolaurate, polyoxy:polyoxyethylene stearate, polyoxypropylene 15 stearyl ether, sodium cholate, sodium glycocholate, sodium taurocholate, sodium glycodeoxycholate, sodium taurodeoxycholate, sodium ursodeoxycholate, taurocholic acid, taurodeoxycholic acid, TWEEN 20, urosdeoxycholic acid, and derivatives, esters, salts and mixtures thereof.

Suitable anti-irritants that can be used in the context of the present embodiments include, for example, steroidal and non-steroidal anti-inflammatory agents or other materials such as menthol, aloe vera, chamomile, alpha-bisabolol, cola nitida extract, green tea extract, tea tree oil, licoric extract, allantoin, caffeine or other xanthines, glycyrrhizic acid and its derivatives.

Suitable preservatives that can be used in the context of the present embodiments include, without limitation, one or more alkanols, parabens such as methylparaben and propylparaben, propylene glycols, sorbates, urea derivatives such as diazolindinyl urea, or any combinations thereof. Additional suitable preservatives include, but are not limited to, benzalkonium chloride, cetrimonium, sodium perborate, stabilized oxvchloro complex, SofZia (Alcon), polyquaternium-1, chlorobutanol, edetate disodium, and polyhexamethylene biguanide.

Further additional ingredients that can be beneficially included in a formulation as described herein include, for example, a disinfecting agent, a tonicity adjusting agent, a stabilizer (stabilizing agent), solubilizing agents, anti-oxidants, and surfactants.

Exemplary suitable disinfecting agents include, but are not limited to, polymeric biguanides, polymeric quarternary ammonium compounds, chlorites, bisbiguanides, chlorite compounds (e.g. potassium chlorite, sodium chlorite, calcium chlorite, magnesium chlorite, or mixtures thereof), and a combination thereof.

A “tonicity adjusting agent” is an agent introduced into an ophthalmic formulation as described herein to reduce local irritation by preventing osmotic shock at the site of application. In some instances, buffer solution and/or a pH adjusting agent that broadly maintains the ophthalmic solution at a particular ion concentration and pH are considered as tonicity adjusting agents. Alternatively or in addition, suitable tonicity adjusting agents include, but are not limited to, various salts, such as halide salts of a monovalent cation, for example, sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, and/or dextrose, mannitol, sorbitol, dextrose, sucrose, urea, propylene glycol, glycerin, trehalose, or a combination thereof.

Examples of suitable solubilizers include citric acid, ethylenediamine-tetraacetate, sodium meta-phosphate, succinic acid, urea, cyclodextrin, polyvinylpyrrolidone, diethylammonium-ortho-benzoate, micelle-forming solubilizers, SPANS, polyoxyethylene sorbitan fatty acid ester, polyoxyethylene n-alkyl ethers, n-alkyl amine n-oxides, poloxamers, phospholipids and cyclodextrins, or combinations thereof. Embodiments of the composition that include a solubilizer that is an irritating penetration enhancer as listed above, include that solubilizer in an amount less than 0.05% by weight of the composition.

According to some of any of the embodiments described herein, the ophthalmic formulation further comprises a cyclodextrin.

Cyclodextrins are cyclic oligosaccharides containing 6, 7, or 8 glucopyranose units, referred to as alpha-cyclodextrin, beta-cyclodextrin, or gamma-cyclodextrin respectively. Cyclodextrins have a hydrophilic exterior, which enhances water-solubility, and a hydrophobic interior which forms a cavity. In an aqueous environment, hydrophobic portions of other molecules often enter the hydrophobic cavity of cyclodextrin to form inclusion compounds. Additionally, cyclodextrins are also capable of other types of nonbonding interactions with molecules that are not inside the hydrophobic cavity. Accordingly, in some embodiments, cyclodextrins are included to increase the solubility and/or stability of the active agent within the formulations described herein. Additionally or alternatively, cyclodextrins serve as controlled release excipients within the formulations described herein.

Exemplary cyclodextrins for use include, without limitation, alpha-cyclodextrin, beta-cyclodextrin, gamma-cyclodextrin, hydroxyethyl-beta-cyclodextrin, hydroxypropyl-gamma-cyclodextrin, sulfated beta-cyclodextrin, sulfated alpha-cyclodextrin, and sulfobutyl ether beta-cyclodextrin.

Other stabilizers that are suitable for use include, for example, fatty acids, fatty alcohols, alcohols, long chain fatty acid esters, long chain ethers, hydrophilic derivatives of fatty acids, polyvinyl pyrrolidones, polyvinyl ethers, polyvinyl alcohols, hydrocarbons, hydrophobic polymers, moisture-absorbing polymers, glycerol, methionine, monothioglycerol, EDTA, ascorbic acid, polysorbate 80, polysorbate 20, arginine, heparin, dextran sulfate, cyclodextrins, pentosan polysulfate and other heparinoids, divalent cations such as magnesium and zinc, or combinations thereof.

Additional useful stabilization agents include one or more anti-aggregation additives to enhance stability of ophthalmic formulations, such as, but not limited to, urea, guanidinium chloride, simple amino acids such as glycine or arginine, sugars, polyalcohols, polysorbates, polymers such as polyethylene glycol and dextrans, alkyl saccharides, such as alkyl glycoside, and surfactants.

Ophthalmically acceptable antioxidants include, by way of example only, ascorbic acid, methionine, sodium thiosulfate and sodium metabisulfite. In one embodiment, antioxidants are selected from metal chelating agents, thiol containing compounds and other general stabilizing agents.

Ophthalmically acceptable surfactants include, but are not limited to, polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40, natural and synthetic lipophilic agents, e.g., phospholipids, cholesterol, and cholesterol fatty acid esters and derivatives thereof; nonionic surfactants, which include for example, polyoxyethylene fatty alcohol esters, sorbitan fatty acid esters (Spans), polyoxyethylene sorbitan fatty acid esters (e.g., polyoxyethylene (20) sorbitan monooleate (Tween 80), polyoxyethylene (20) sorbitan monostearate (Tween 60), polyoxyethylene (20) sorbitan monolaurate (Tween 20) and other Tweens, sorbitan esters, glycerol esters, e.g., Myrj and glycerol triacetate (triacetin), polyethylene glycols, cetyl alcohol, cetostearyl alcohol, stearyl alcohol, polysorbate 80, polaxamers, poloxamines, polyoxyethylene castor oil derivatives and other Cremophors, sulfosuccinates, alkyl sulphates (SLS); PEG glyceryl fatty acid esters such as PEG-8 glyceryl caprylate/caprate (Labrasol), PEG-4 glyceryl caprylate/caprate (Labrafac Hydro WL 1219), PEG-32 glyceryl laurate (Gelucire 444/14), PEG-6 glyceryl mono oleate (Labrafil M 1944 CS), PEG-6 glyceryl linoleate (Labrafil M 2125 CS); propylene glycol mono- and di-fatty acid esters, such as propylene glycol laurate, propylene glycol caprylate/caprate; ascorbyl-6-palmitate, stearylamine, sodium lauryl sulfate, polyoxethyleneglycerol triiricinoleate, and any combinations or mixtures thereof; anionic surfactants include, but are not limited to, calcium carboxymethylcellulose, sodium carboxymethylcellulose, sodium sulfosuccinate, dioctyl, sodium alginate, alkyl polyoxyethylene sulfates, sodium lauryl sulfate, triethanolamine stearate, potassium laurate, bile salts, and any combinations or mixtures thereof; and d) cationic surfactants such as cetyltrimethylammonium bromide, and lauryldimethylbenzyl-ammonium chloride.

Any of the additional ingredients or agents described herein is preferably selected as being compatible with the components of the formulations as described herein, such that there is no interference with the availability of at least the active agent in the formulation.

Any of the additional ingredients described herein is further preferably selected as being biocompatible, and more preferably as ophthalmically acceptable.

Exemplary formulations according to some of the present embodiments includes an ophthalmically acceptable carrier as described herein in any of the respective embodiments and one or more additional agent(s) selected from any of the respective embodiments as described herein and any combination thereof.

Formulations of the present embodiments may, if desired, be presented in a pack or dispenser device, such as an FDA (the U.S. Food and Drug Administration) approved kit, which may contain one or more unit dosage forms containing the formulation. The pack or dispenser device may be accompanied by instructions for administration. The pack or dispenser may also be accompanied by a notice associated with the container in a form prescribed by a governmental agency regulating the manufacture, use or sale of pharmaceuticals, which notice is reflective of approval by the agency of the form of the compositions for human or veterinary administration. Such notice, for example, may be of labeling approved by the U.S. Food and Drug Administration for prescription drugs or of an approved product insert. Compositions of the invention formulated in a compatible pharmaceutical carrier may also be prepared, placed in an appropriate container, and labeled for a medical indication, as detailed herein.

The ophthalmic formulations described herein may be packed or presented in any convenient way. For example, they may be packed in a tube, a bottle, a dispenser, a squeezable container, or a pressurized container, using techniques well known to those skilled in the art and as set forth in reference works such as Remington’s Pharmaceutical Science 15^{th} Ed. It is preferred that the packaging is done in such a way so as to minimize contact of the unused compositions with the environment, in order to minimize contamination of the formulation before and after the container is opened.

The formulations described herein are preferably supplied in the concentration intended for use but may also be prepared as concentrates that are diluted prior to use. For example, concentrates requiring dilution ratios of 2:1 to 100:1 parts carrier to a concentrate are contemplated, including any intermediate values and subranges therebetween.

According to some of any of the embodiments described herein, and depending on the form and/or type of the ophthalmic formulation, a concentration of the PAR-1 antagonist or the agent (e.g., peptide conjugate as described herein) is lower than 1 molar, or lower than 800 millimolar, or lower than 500 millimolar, or lower than 100 millimolar, or lower than 1 millimolar, or lower than 500 nanomolar, or lower than 100 nanomolar, or lower than 1 nanomolar. In exemplary embodiments, an amount of the active agent (the PAR-1 antagonist or the agent (e.g., peptide conjugate as described herein)) ranges from 1 picomolar to 500 millimolar, from 1 picomolar to 100 millimolar, from 1 picomolar to 1 millimolar, or from 1 picomolar to 500 nanomolar, or from 1 picomolar to 100 nanomolar, or from a picomolar to 500 picomolar, or from a picomolar to 100 picomolar, or from 1 nanomolar to 500 nanomolar, or from 1 nanomolar to 100 nanomolar, including any intermediate values and subranges therebetween.

According to exemplary embodiments, the ophthalmic formulation is an aqueous formulation as described herein in any of the respective embodiments, and comprises the active agent at a concentration as described herein in any of the respective embodiments, for example, in a range of from 1 picomolar to 500 millimolar, or from 1 picomolar to 100 millimolar, or from 1 picomolar to 1 millimolar, or from 1 picomolar to 500 nanomolar, or from 1 picomolar to 100 nanomolar, or from a picomolar to 500 picomolar, or from a picomolar to 100 picomolar, or from 1 nanomolar to 500 nanomolar, or from 1 nanomolar to 100 nanomolar, including any intermediate values and subranges therebetween.

In some embodiments, the formulation described herein is packaged in a packaging material and identified in print, in or on the packaging material, for use in treating or preventing retinal degeneration, or for any of the uses described herein in any of the respective embodiments and any combination thereof.

According to any of the embodiments described herein, an ophthalmic formulation can be considered interchangeably as a pharmaceutical composition formulated for topical application to an eye of a subject or for ophthalmic administration, or as an ophthalmic composition.

According to some of any of the embodiments described herein, the ophthalmic formulation is in a form of eye drops, for example, in a form of a liquid solution or emulsion (e.g., microemulsion), which can be packaged in a device or dispenser configured to dispensed drops.

### Articles-of-Manufacturing

According to an aspect of some embodiments of the present invention, an article-of-manufacturing is provided, which comprises the formulation or the composition as described herein in any of the respective embodiments, and any combination thereof, and means for topically applying the formulation to the treated area, or to contact the treated area with the formulation. In some embodiments, the article-of-manufacturing is configured to apply the formulation to, or contact the formulation with, an eye of a subject.

In some embodiments, the article-of-manufacturing comprises the formulation as described herein, in a form of a liquid (e.g., solution or suspension or emulsion), packaged in a container, and means for applying the composition as drops, spray, aerosol, foam, using techniques well known to those skilled in the art and as described herein.

In some embodiments, the article-of-manufacturing comprises the formulation as described herein, in a form of a cream, paste, ointment, gel and the likes, packaged in a suitable container, and optionally comprising means for dispensing the formulation from the container, for applying it to, or contact it with, an eye of a subject.

In some embodiments, the article-of-manufacturing comprises the formulation as described herein, incorporated in and/or on a solid substrate, as described herein. The composition can be packaged in a sterile packaging.

The article-of-manufacturing can be labeled as described herein, for example, by being identified in print, in or on the packaging material, for use in treating or preventing any of the medical conditions as described herein.

According to some embodiments, the article-of-manufacturing is an ophthalmic delivery device or system. In some of these embodiments, the ophthalmic delivery device or system is configured for controlled sustained release of the active agent.

Exemplary ophthalmic delivery devices or systems include, but are not limited to, systems such as particles (for example, nanoparticles and/or microparticles) for controlled release of the active agent, including, but not limited to, liposomes, niosomes, discosomes and dendrimers; ocular systems in a form of, for example, minidiscs, minitablets, and other non-invasive delivery devices such as, for example, a topical ophthalmic drug delivery device (TODD) or a contact lens (onto which the formulation can be applied, or included within the contact lens in a releasable form).

Exemplary ophthalmic delivery devices or systems include, but are not limited to, ocular inserts such as, for example, soluble ophthalmic drug inserts, artificial tear inserts, collagen shields, a punctal plug, a scleral patch, a scleral ring, a Cul-de sac insert, a subconjunctival/episcleral implant, and an intravitreal implant.

The ophthalmic delivery device or system can be a biodegradable ophthalmic delivery device or system or a non-biodegradable ophthalmic delivery device or system.

An exemplary ophthalmic delivery device comprises a core or reservoir which comprises a formulation as described herein and is configured for a controlled sustained release of the active agent. In some of these embodiments, the formulation is in a form of a solution, a gel, or in a solid form, as described herein. In other embodiments, the formulation is dispersed (e.g., uniformly) in or on the material of the ophthalmic delivery device, and can be configured for a controlled sustained release of the active agent.

### Uses

According to an aspect of some embodiments of the present invention there are provided methods and uses of an ophthalmic formulation or an article-of-manufacturing comprising same, as described herein in any of the respective embodiments and any combination.

According to an aspect of some embodiments of the present invention there is provided a method of treating an ocular condition (an ocular disease or disorder), which comprises ophthalmic administration of an ophthalmic formulation as described herein. The ophthalmic administration can comprise topical application of the formulation to an eye of a subject in need thereof or otherwise contacting the formulation with the eye of the subject.

According to an aspect of some embodiments of the present invention there is provided an ophthalmic formulation or an article-of-manufacturing comprising same, as described herein in any of the respective embodiments and any combination, for use in treating an ocular condition as described herein. The formulation or article-of-manufacturing are used by topical application of the formulation to an eye of a subject in need thereof or otherwise contacting the formulation with the eye of the subject.

According to an aspect of some embodiments of the present invention there is provided an ophthalmic formulation as described herein in any of the respective embodiments and any combination, for use as a medicament, or for use in a preparation of medicament. In some embodiments, the medicament is for treating an ocular condition as described herein. The medicament is used by topical application of the formulation to an eye of a subject in need thereof or otherwise contacting the formulation with the eye of the subject.

In some of any of the embodiments described herein, any of the ophthalmic formulations or compositions or articles-of-manufacturing comprising same are usable, or are for use, in treating medical conditions that affect a retinal tissue in a subject (retinal diseases or disorders). The formulation or article-of-manufacturing are used by topical application of the formulation to an eye of a subject in need thereof or otherwise contacting the formulation with the eye of the subject.

In some of any of the embodiments described herein, any of the ophthalmic formulations or compositions or articles-of-manufacturing comprising same are usable, or are for use, in treating ocular conditions that are associated with PAR1 overexpression and/or over-activity, or which are treatable by downregulating an expression or activity of PAR1 in an ocular tissue, preferably a retinal tissue (e.g., in photoreceptor cells in the retina).

In some of any of the embodiments described herein, any of the ophthalmic formulations or compositions or articles-of-manufacturing comprising same are usable, or are for use, in treating a disease or disorder associated with overexpression and/or overactivity of PAR1 in a retinal tissue (e.g., in photoreceptor cells in the retina) of a subject.

In some of any of the embodiments described herein, any of the ophthalmic formulations or compositions or articles-of-manufacturing comprising same are usable, or are for use, in treating a disease or disorder treatable by downregulating an expression and/or activity of PAR1 in a retinal tissue (e.g., in photoreceptor cells in the retina) of a subject.

In some of any of the embodiments described herein, any of the ophthalmic formulations or compositions or articles-of-manufacturing comprising same are usable, or are for use, in treating a disease or disorder treatable by interfering with a PAR1/protease interaction in a retinal tissue (e.g., in photoreceptor cells in the retina) of a subject. In some embodiments, the protease is as described herein, for example, thrombin.

Retinal diseases or disorders that are associated with PAR1 overexpression and/or overactivity include, but are not limited to, retinal degeneration, retinal dystrophy, retinal inflammation and abnormal proliferation (e.g., retinal tumors), and any pathology or medical condition that involves retinal inflammation and/or neovascularization.

In some of any of the embodiments described herein, any of the ophthalmic formulations or compositions or articles-of-manufacturing comprising same are usable, or are for use, in treating, slowing, reducing, arresting or preventing retinal degeneration in a subject in need thereof.

In some of any of the embodiments described herein, any of the ophthalmic formulations or compositions or articles-of-manufacturing comprising same are usable, or are for use, in treating a condition that may lead to vision deterioration or loss in a subject in need thereof.

Herein, the term “subject” includes mammals, preferably warm-blooded mammals including birds, cows, horses, goat, sheep, pigs, dogs, cats, chickens and turkeys, and more preferably human beings at any age, which suffer from, or are susceptible to suffer from, an ocular condition as described herein, for example, a pathology that requires treating, slowing, reducing, arresting or preventing retinal degeneration and/or a pathology that may lead to vision deterioration or loss.

Herein and in the art, the phrase “retinal tissue” describes a layer of nervous tissue that covers the inside of the back two-thirds of the eyeball, in which stimulation by light occurs, initiating the sensation of vision. The retinal tissue is the innermost, light-sensitive layer of tissue of the eye of most vertebrates. The neural retina consists of several layers of neurons interconnected by synapses and is supported by an outer layer of pigmented epithelial cells. The primary light-sensing cells in the retina are the photoreceptor cells, which include rods and cones. Rods function mainly in dim light and provide black-and-white vision. Cones function in well-lit conditions and are responsible for the perception of color, as well as high-acuity vision used for tasks such as reading. Other retinal cells include bipolar cells, retinal ganglion cells, horizontal cells and amacrine cells.

The phrase “retinal degeneration” describes a retinopathy which is reflected by deterioration of the retina caused by the progressive death of its cells.

Retinal degeneration is associated with, or typically caused by, artery or vein occlusion, diabetic retinopathy, retrolental fibroplasia, retinopathy of prematurity, or a disease, usually hereditary, such as macular degeneration (e.g., age-related macular degeneration) or retinitis pigmentosa (RP).

Retinal degeneration is typically presented by one or more of impaired vision, night blindness, retinal detachment, light sensitivity, tunnel vision, and loss of peripheral vision to total loss of vision.

In some embodiments, the retinal degeneration is associated with diabetic retinal neuropathy. In these embodiments, the subject is a diabetic subject who is afflicted with retinal neuropathy or is susceptible to be afflicted with retinal neuropathy.

In some embodiments of any of the methods and uses described herein, the treatment comprises topical administration of the formulation to, or contacting the formulation with, an eye of the subject.

Topical administration (ophthalmic administration) can be performed using any of the formulations, systems, devices or articles as described herein in any of the respective embodiments.

Contacting the formulation with the eye can be effected, for example, by contacting the eye with a device or article onto which the formulation is deposited, or from which the formulation can be released (e.g., controllably released).

In some of any of the embodiments described herein, the formulation of the present embodiments is used in an effective amount that provides a desired prophylactic, therapeutic or pharmaceutical effect. Determination of the effective amount, and consequently the dose and dose frequency (regimen), is within the capability of one skilled in the art, in light of the disclosure provided herein. Generally, medical personnel such as a doctor prescribing a pharmaceutical composition for use in accordance with the teachings of the invention prescribe a dosage regime including one or more administrations of a dose of the formulation over a period of time (e.g., once a day, twice a day, three times a day). The dosage regime is generally chosen to be effective, that is to say sufficient to achieve a desired beneficial effect, e.g., to treat a condition as described herein.

Determination of an effective dosage regime is within the capability of a person having ordinary skill in the art in light of the disclosure provided herein for example using techniques with which one of average skill is familiar, which are discussed in numerous reference works, such as Remington’s Pharmaceutical Science 15th Edition. Factors in determining the dosage regime vary with the type of the condition as well as such factors as the concentration of the active agent, the subject being treated, the severity of the condition, the age, body weight and response of an individual patient and the judgment of the prescribing physician.

In exemplary embodiments, the formulation is used by ophthalmologically (ophthalmically) administering to the subject 1 to 50, or 1 to 30, or 1 to 20, or 1 to 10, or 5 to 20, or 5 to 10, drops of a liquid formulation (e.g., an aqueous formulation), from 1 to 5 times a day. In some of these embodiments, a concentration of the active agent in the formulation in as described herein in any of the respective embodiments and any combination thereof. The formulation can be administered every day, or 2-3 times a week, or once a week, and can be administered for a determined time period (e.g., until the condition to be treated is improved or symptoms are ameliorated), or chronically, depending on the condition to be treated and its severity.

As used herein the term “about” refers to ± 10 % or ± 5 %.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

The term “hydrocarbon”, as used herein, encompasses any moiety that is based on a linear and/or cyclic chain of carbons which are mainly substituted by hydrogens. A hydrocarbon can be a saturated or unsaturated moiety, and can optionally be substituted by one or more substituents, as described herein.

The term “alkyl” describes a saturated aliphatic hydrocarbon including straight chain and branched chain groups. Preferably, the alkyl group has 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. More preferably, the alkyl is a medium size alkyl having 2 to 10 carbon atoms. Most preferably, unless otherwise indicated, the alkyl is a lower alkyl having 2 to 6 carbon atoms. The alkyl group may be substituted or unsubstituted, as defined herein.

The term “cycloalkyl” or “alicyclic” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. The heteroalicyclic may be substituted or unsubstituted. Representative examples are piperidine, piperazine, tetrahydrofurane, tetrahydropyrane, morpholino and the like.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furane, thiophene, imidazole, oxazole, thiazole, pyrazole, pyridine, pyrimidine, quinoline, isoquinoline and purine. The heteroaryl group may be substituted or unsubstituted.

Whenever an alkyl, cycloalkyl, heteroalicyclic, aryl, heteroaryl or a hydrocarbon is substituted by one or more substituents, each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

A “hydroxy” group refers to an —OH group.

An “azide” group refers to a —N═N⁺═N⁻ group.

An “alkoxy” group refers to both an -O-alkyl and an -O-cycloalkyl group, as defined herein.

An “aryloxy” group refers to both an -O-aryl and an -O-heteroaryl group, as defined herein.

A “thiohydroxy” or “thiol” group refers to a —SH group.

A “thioalkoxy” group refers to both an -S-alkyl group, and an -S-cycloalkyl group, as defined herein.

A “thioaryloxy” group refers to both an -S-aryl and an -S-heteroaryl group, as defined herein.

A “carbonyl” group refers to a —C(═O)—R′ group, where R′ is defined as hereinabove. An acetyl is a carbonyl, as defined herein, wherein R′ is a substituted or unsubstituted methyl.

A “thiocarbonyl” group refers to a —C(═S)—R′ group, where R′ is as defined herein.

A “C-carboxy” group refers to a —C(═O)—O—R′ groups, where R′ is as defined herein.

An “O-carboxy” group refers to an R′C(═O)—O— group, where R′ is as defined herein.

An “oxo” group refers to a ═O group.

A “carboxylate” or “carboxyl” encompasses both C-carboxy and O-carboxy groups, as defined herein.

A “carboxylic acid” group refers to a C-carboxy group in which R′ is hydrogen.

A “thiocarboxy” or “thiocarboxylate” group refers to both —C(═S)—O—R′ and —O—C(═S)R′ groups.

An “ester” refers to a C-carboxy group wherein R′ is not hydrogen.

An ester bond refers to a —O—C(═O)— bond.

A “halo” group refers to fluorine, chlorine, bromine or iodine.

A “sulfinyl” group refers to an —S(═O)—R′ group, where R′ is as defined herein.

A “sulfonyl” group refers to an —S(═O)₂—R′ group, where R′ is as defined herein.

A “sulfonate” group refers to an —S(═O)₂—O—R′ group, where R′ is as defined herein.

A “sulfate” group refers to an —O—S(═O)₂—O—R′ group, where R′ is as defined as herein.

A “sulfonamide” or “sulfonamido” group encompasses both S-sulfonamido and N-sulfonamido groups, as defined herein.

An “S-sulfonamido” group refers to a —S(═O)₂—NR′R″ group, with each of R′ and R″ as defined herein.

An “N-sulfonamido” group refers to an R′S(═O)₂—NR″ group, where each of R′ and R″ is as defined herein.

An “O-carbamyl” group refers to an —OC(═O)—NR′R″ group, where each of R′ and R″ is as defined herein.

An “N-carbamyl” group refers to an R′OC(═O)—NR″— group, where each of R′ and R″ is as defined herein.

A “carbamyl” or “carbamate” group encompasses O-carbamyl and N-carbamyl groups.

A carbamate bond describes a —O—C(═O)—NR′— bond, where R′ is as described herein.

An “O-thiocarbamyl” group refers to an —OC(═S)—NR′R″ group, where each of R′ and R″ is as defined herein.

An “N-thiocarbamyl” group refers to an R′OC(═S)NR″— group, where each of R′ and R″ is as defined herein.

A “thiocarbamyl” or “thiocarbamate” group encompasses O-thiocarbamyl and N-thiocarbamyl groups.

A thiocarbamate bond describes a —O—C(═S)—NR′— bond, where R′ is as described herein.

A “C-amido” group refers to a —C(═O)—NR′R″ group, where each of R′ and R″ is as defined herein.

An “N-amido” group refers to an R′C(═O)—NR″ — group, where each of R′ and R″ is as defined herein.

An “amide” group encompasses both C-amido and N-amido groups.

An amide bond describes a —NR′—C(═O)— bond, where R′ is as defined herein.

A “urea” group refers to an —N(R′)—C(═O)—NR″R′” group, where each of R′ and R″ is as defined herein, and R″’ is defined as R′ and R″ are defined herein.

A “nitro” group refers to an —NO₂ group.

A “cyano” group refers to a —C≡N group.

The term “phosphonyl” or “phosphonate” describes a —P(═O)(OR′)(OR″) group, with R′ and R″ as defined hereinabove.

The term “phosphate” describes an —O—P(═O)(OR′)(OR″) group, with each of R′ and R″ as defined hereinabove.

A “phosphoric acid” is a phosphate group is which each of R is hydrogen.

The term “phosphinyl” describes a —PR′R″ group, with each of R′ and R″ as defined hereinabove.

The term “thiourea” describes a —N(R′)—C(═S)—NR″— group, with each of R′ and R″ as defined hereinabove.

Herein throughout, R, R′ and R″ are each independently hydrogen, alkyl, cycloalkyl, or aryl, as these terms are defined herein, and can alternatively be each independently hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine, as these terms are defined herein.

As used herein, the term “alkylene glycol” describes a —O—[(CR′R″)_{z}—O]_{y}—R‴ end group or a —O—[(CR′R″)_{z}—O]_{y}— linking group, with R′, R″ and R‴ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo(alkylene glycol). Any of the compounds (e.g., active agents, compound of Formula I) described herein can be in a form of a pharmaceutically acceptable salt thereof.

The phrase “pharmaceutically acceptable salt” refers to a charged species of the parent compound and its counter ion, which is typically used to modify the solubility characteristics of the parent compound and/or to reduce any significant irritation to an organism by the parent compound, while not abrogating the biological activity and properties of the administered compound.

The present invention further encompasses prodrugs, solvates and hydrates of the substances described herein.

As used herein, the term “prodrug” refers to an agent, which is converted into the active compound (the active parent drug) in vivo. Prodrugs are typically useful for facilitating the administration of the parent drug. They may, for instance, be bioavailable by oral administration whereas the parent drug is not. The prodrug may also have improved solubility as compared with the parent drug in pharmaceutical compositions. Prodrugs are also often used to achieve a sustained release of the active compound in vivo. An example, without limitation, of a prodrug would be a compound, as described herein, having one or more carboxylic acid moieties, which is administered as an ester (the “prodrug”). Such a prodrug is hydrolysed in vivo, to thereby provide the free compound (the parent drug). The selected ester may affect both the solubility characteristics and the hydrolysis rate of the prodrug.

The term “solvate” refers to a complex of variable stoichiometry (e.g., di-, tri-, tetra-, penta-, hexa-, and so on), which is formed by a solute (the compound as described herein) and a solvent, whereby the solvent does not interfere with the biological activity of the solute. Suitable solvents include, for example, ethanol, acetic acid and the like.

The term “hydrate” refers to a solvate, as defined hereinabove, where the solvent is water.

The present embodiments further encompass any enantiomers and diastereomers of the compounds described herein.

As used herein, the term “enantiomer” refers to a stereoisomer of a compound that is superposable with respect to its counterpart only by a complete inversion/reflection (mirror image) of each other. Enantiomers are said to have “handedness” since they refer to each other like the right and left hand. Enantiomers have identical chemical and physical properties except when present in an environment which by itself has handedness, such as all living systems. In the context of the present embodiments, a compound may exhibit one or more chiral centers, each of which exhibiting an R- or an S-configuration and any combination, and compounds according to some embodiments of the present invention, can have any their chiral centers exhibit an R- or an S-configuration.

The term “diastereomers”, as used herein, refers to stereoisomers that are not enantiomers to one another. Diastereomerism occurs when two or more stereoisomers of a compound have different configurations at one or more, but not all of the equivalent (related) stereocenters and are not mirror images of each other. When two diastereoisomers differ from each other at only one stereocenter they are epimers. Each stereo-center (chiral center) gives rise to two different configurations and thus to two different stereoisomers. In the context of the present invention, embodiments of the present invention encompass compounds with multiple chiral centers that occur in any combination of stereo-configuration, namely any diastereomer.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Material and Experimental Methods

Animals: 13-week-old male C57BL/6JOlaHsd mice (purchased from Envigo Laboratories, Israel) and male PAR1 knockout C57BL/6J (PAR1^{-/-}) were used and housed at the Sheba Medical Center animal facility. All animal procedures and experiments were approved by the Institutional Animal Care Committee at the Sheba Medical Center, Tel-Hashomer (1210/19-ANIM) and conformed to recommendations of the Association for Research in Vision and Ophthalmology Statement for the Use of Animals in Ophthalmic and Vision Research and according to ARRIVE (Animal Research: Reporting of In Vivo Experiments) guidelines.

Paraformaldehyde (PFA) Perfusion: Wherever indicated, mice were perfused before eye removal. Mice were anesthetized with an intraperitoneal injection (IP) of 75 mg/kg ketamine and 10 mg/kg xylazine, and were perfused transcardially with PBS pH 7.4, followed by 4% paraformaldehyde (PFA, Sigma-Aldrich, P6148) in 0.1 M phosphate buffer (pH 7.4). Eyes were removed, fixed in 4 % formaldehyde, and processed as indicated herein.

Diabetes induction: Diabetes was induced in mice at age of 8 weeks by a single intraperitoneal injection of 150 mg/Kg STZ (Sigma-Aldrich). Blood glucose concentration was measured from the tail vein using Xpress-i glucometer (Nova Biomedical). Hyperglycemia was defined as blood glucose >200 ml/dL [Shavit-Stein et al. (2019) PLoS One 14, e0219453].

Systemic coagulation: Prothrombin time (PT), activated partial thromboplastin time (aPTT) and thrombin time (TT) were measured in mice plasma samples using an ACLTOP® 500 autoanalyzer at Sheba Medical Center MegaLab according to standard operating procedures [Shavit-Stein et al. (2018) Front. Neurol. 9, 1087].

Electroretinogram (ERG): Mice were dark-adapted for 16 hours. ERG responses to light flashes at 5 increasing intensities (0.023, 0.249, 2.44, 7.8 and 23.5 cd-s/m²) are recorded from both eyes simultaneously. Light-adapted ERG is similarly performed following 5 minutes light adaptation [Edelshtain et al. (2019) Algal Research 43, 101607].

Co-localization analysis: Co-localization analysis was performed using Image Zen software (LSM software ZEN 2012, Zeiss, Germany) on three separate regions of interest (ROI) in retinal sections of two mice. Results are presented as Pearson’s Correlation Coefficients of merged fluorescence histograms after creating a uniform region of interest (ROI) for each protein.

Neuroretina isolation: Mice were euthanized by IP injection of pentobarbital, 100 µl (Pentobarbital 20 %, CTS, Israel). Eyes are removed and placed in ice-cold PBS. Corneas are removed by punching a hole in the limbus using a 25-G needle, followed by an incision around the periphery of the cornea. The lens was removed, and the neuroretina was separated from the RPE and the sclera using two pairs of blunt tip tweezers. The procedure was performed on ice under a stereo microscope (SMZ745T, Nikon, Japan).

Neuroretina and platelets preparation: Mouse neuroretinas were separated from the posterior eye segment and were homogenized in radioimmunoprecipitation assay (RIPA) buffer (50 mM Tris HCl, pH 7.6, 150 mM NaCl, 1% NP-40, 0.5% Sodium Deoxycholate and 0.1% SDS supplemented with commercial Protease Inhibitor Cocktail (P-2714, Sigma-Aldrich, Saint Louis, MO, USA) using a beads-based homogenizer (BB*24B, Next Advance, USA). For Platelets preparation, blood was collected into tubes containing CPDA1 (citric acid, sodium citrate, monobasic sodium phosphate, dextrose, and adenine). Blood was centrifuged (100 g, 9 minutes) to separate platelets rich plasma (PRP). Residual erythrocytes were removed by centrifugation (100 g, 6 minute). The PRP was centrifuged (1000 g, 5 minutes) to sediment the platelets and the supernatant was removed. Platelets were re-suspended in PBS and were centrifuged (1000 g, 3 minutes) to wash the platelets and remove residual plasma. Platelets were re-suspended in PBS for protein concentration determination and further western blot analysis. A bicinchoninic acid (BCA) kit (QPRO-BCA kit, PRTD1,0500, Cyanagen, Italy) was used to determine protein concentration.

Thrombin activity in the neuroretina: Thrombin activity was assessed according to Gera et al. [(2016) Neuroscience 339, 587-598], using a specific fluorogenic thrombin substrate (ex.360/em.465 nm). Neuroretinas were removed from mice at selected time points and were placed in a black 96-well microplate in the presence of endopeptidase inhibitors to eliminate the effect of widely abundant CNS endopeptidases on the assay. Known bovine thrombin concentrations were used as to create a calibration curve for each experiment.

Multicolor fundus imaging and SD-OCT: Retinal structure was determined in anesthetized mice following pupil dilation using a Heidelberg Spectralis SD-OCT employing TruTrack active eye tracking and AutoRescan procedures for rescanning the retina at the exact same location as the baseline examinations, as described in Bubiset al. [(2019) Transl Vis Sci Technol 8, 26].

Immunofluorescence staining: Mice were sacrificed at 13 weeks of age, and their eyes were fixed in 4 % formaldehyde. Retinal paraffin sections were deparaffinized and rehydrated. Following epitope retrieval using citrate buffer (pH 6.0, Zytomed Systems GMBH, Germany), retinal sections were blocked with 10 % donkey serum in phosphate buffered saline (PBS) containing 0.1 % Triton x-100 followed by incubation with primary antibodies (diluted in PBS containing 0.1 % Triton x-100 and 1 % donkey serum). Next, sections were extensively washed in PBS and incubated with fluorescently labeled secondary antibodies. Sections were then mounted with aqueous mounting medium containing 4′,6-diamidino-2-phenylindole (DAPI Fluoromount-G, Emsidiasum), and are viewed with a confocal microscope (Confocal Microscope ZEISS LSM 700).

Histology and Immunofluorescence analysis: Retinal and optic nerve sections (4 µm) were stained with hematoxylin/eosin to evaluate gross structural changes. Immunofluorescence analysis is performed to characterize the expression pattern of PAR1 in mouse and human neuroretina using specific antibodies for PAR1 and co-staining with neuroretinal nuclear markers (e.g. PAX6 and OTX2 transcription factors), neuroretinal cell type specific markers (rhodopsin and cone opsins for photoreceptors, glutamine synthetase for Muller cells, PKCα for bipolar cells and Brn3a for retinal ganglion cells. The effect of PARIN5 eye drops treatment on neuroretinal apoptosis is assessed by TUNEL and caspase-3 staining and microglia activation and MÜller cell gliosis is evaluated by Iba-1, Kir4.1 and GFAP staining.

Western blotting: Neuroretinas and platelets were collected from C57BL/6J WT and PAR1⁻ ^{/-} mice for Western blot analysis.

In one set of experiments, eyes from different mouse groups were snap frozen, the neuro-retina was removed from the eye cups and lysed. To determine subcellular localization of PAR1, nuclear/cytoplasmic fractionation was performed using nuclear extraction kit (Active motif). Western blots with PAR1, thrombin, FXa, GFAP, Iba-1, PN1, APP and ATII antibodies were performed with β-actin as a loading control, LaminB1 and α-tubulin were used as controls for nuclear/cytoplasmic fractionation.

In another set of experiments, 20 micrograms of total protein samples from the neuroretinas and 5 micrograms of total proteins from platelets were separated by polyacrylamide gel electrophoresis and transferred onto nitrocellulose membranes for western blot analysis. Membranes were incubated overnight at 4° C. with primary mouse anti-PAR1 antibody (1:500, NBP-71770, Nuvos biologicals) in tris-buffered saline (Tris HCl 50 mM, NaCl 150 mM, Tween 0.1 %). Membranes were then washed and incubated at room temperature with horseradish peroxidase-conjugated goat anti-mouse secondary antibodies (Jackson ImmunoResearch Laboratories, USA). Enhanced chemiluminescence (ECL) method (MYECL Imager, Thermo Scientific, USA) was used for protein bands detection.

Human Retina: De-identified formalin-fixed paraffin-embedded retinal sections from non-diabetic donors and DR patients were obtained from the Sheba Institute of Pathology Tissue.

Reverse transcription quantitative real-time PCR (qRT-PCR): Animals were euthanized with pentobarbital. Neuroretinas were separated from the posterior eye segment as indicated herein and were homogenized with a bullet blender homogenizer (BB*24B, Next Advance, USA) at maximal speed for two minutes. RNA was extracted from the neuroretina using RNA, Aurum™ Total RNA Mini Kit (Bio rad Laboratories, 7326820 Hercules, CA, USA) according to the manufacturer’s instructions. One microgram of total RNA was used for reverse transcription using high-capacity cDNA reverse transcription kit (Applied Biosystems AB-4374966 Thermo Fisher Scientific, USA). The qRT-PCR was performed on a StepOne™ Real-Time PCR System (Applied Biosystems, Rhenium, Israel) using Fast SYBR Green Master (Applied Biosystems AB-4385612 Thermo Fisher Scientific, USA). A standard amplification program was used, one cycle of 95° C. for 20 seconds (s) and 40 cycles of 95° C. for 3 seconds and 60° C. for 30 seconds. The results were normalized to a reference gene expression, Hypoxanthine guanine phosphoribosyl transferase (HPRT) within the same cDNA sample and calculated using the 2^ΔCT method. Results are presented as fold changes relative to HPRT and reported as mean ± standard error (SE).

KCl-induced depolarization: High levels of KCl were used for retinal depolarization in order to compare thrombin activity between two halves of mouse retinas under low (5.6 mM) and high (56 mM) KCl conditions in the same microplate.

Statistics: Pearson’s Correlation Coefficients of Co-localization analysis is performed using Image Zen software (LSM software ZEN 2012, Zeiss, Germany). Statistical analysis of thrombin activity results were assessed by paired, two tailed, T test analysis. All calculations were performed using GraphPad Prism 8 (GraphPad Software Inc., California).

### Example 1

### Background Art

The present inventors have previously designed and successfully practiced unique molecules that specifically block the interaction of PAR1 and its activating protease, as schematically shown in FIG. 1. These molecules are peptide conjugates, comprising a peptide portion derived from the specific thrombin-recognition site on PAR1 (the sequence E³⁰SKATNATLDPR⁴¹; SEQ ID NO:9). The peptides are protected at the amino terminus by an amine-protecting group (N-terminus capping moiety) and are conjugated to a protein-disabling moiety such as, for example, a chloromethylketone (CMK) group at the carboxy terminus. See, for example, WO 2015/173802.

The protein-disabling moiety (e.g., CMK) is typically a highly reactive group that is capable of irreversibly inhibiting all potential proteases (such as thrombin, Factor Xa, APC and MMP1) that recognize the specific peptide sequence on PAR1, and this interferes with the protease/PAR1 interaction and prevent the activation of pathways such as the specific thrombin/PAR1 pro-inflammatory pathway, but do not block other anti-inflammatory down-stream pathways induced by PAR1 activation (in comparison to PAR1 antagonist) and do not cause bleeding as direct thrombin inhibitors.

The designed molecules were screened in vitro for their ability to inhibit commercial thrombin and glioma secreted thrombin. In these glioma cell lines, inhibition of thrombin activity by the commercial inhibitor NAPAP and blocking PAR1 activation, changes the cell morphology and significantly inhibits their growth, as described in Shavit-Stein et al. (2018) Front. Neurol. 9, 108.

The designed molecules were also screened for the major potential side effect related to their ability to inhibit coagulation and the associated risk of hemorrhage. In the conducted tests, it was determined that a molecule containing a 5 amino-acid residues sequence (³⁷TLDPR⁴¹-CMK, designated PARIN5) presents a significant inhibition of thrombin together with a promising safety profile, as described, for example, in WO 2015/173802.

### Example 2

### Characterization of Retinal Parameters in Diabetic Mice

STZ-induced diabetic mice were tested for their retinal function by electroretinography (ERG, as described hereinabove).

STZ-induced diabetic mice and control mice were dark adapted for 16 hours and retinal function was measured using ERG. Maximal ERG a-wave response, reflecting rod photoreceptor function was measured in response to increasing light intensities.

As shown in FIG. 2, diabetic mice demonstrated significantly lower a-wave ERG responses (green curve, all p<0.05) compared with control mice (blue curve), indicating a significant reduction in photoreceptor function following diabetes induction.

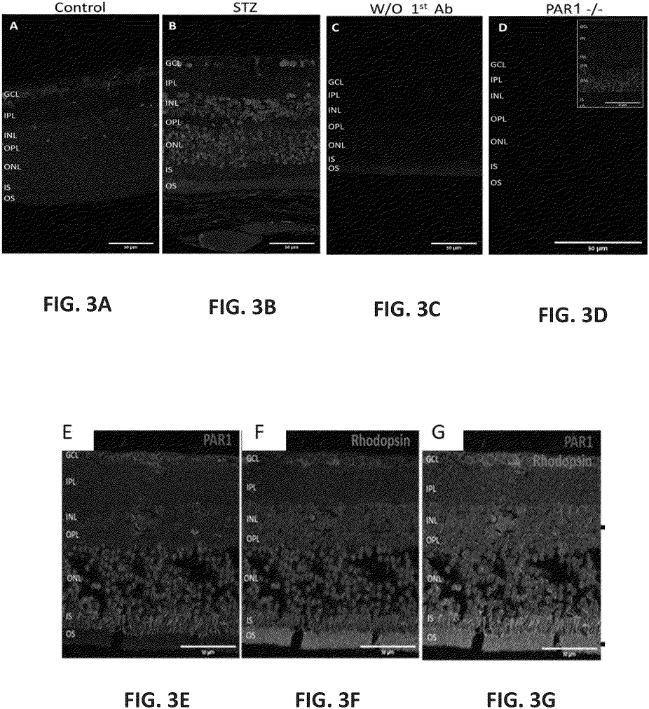

In further studies, retinal sections from control C57BL/6 mice, PAR1 knock-out mice (PAR1^{-/-}) and diabetic mice (following STZ diabetic induction) were fixed in formaldehyde and paraffin sections and were stained with anti-PAR1 antibody and counter stained with 4′,6-diamidino-2-phenylindole (DAPI). The obtained images are shown in FIGS. 3A-3D. As shown in FIGS. 3A and 3B, PAR1 is expressed in retinal inner nuclear layer cells (bipolar cells and amacrine cells), photoreceptor cells and ganglion cells in vivo under physiological conditions. A higher expression level of PAR1 was observed in diabetic mice 5 weeks following STZ diabetes induction and the protein concentration in the nucleus was higher (FIG. 3B). No staining was observed in retinal sections incubated only with secondary antibody (W/O 1^{st} Ab, FIG. 3C) or retinal sections from PAR1 knockout mice (FIG. 3D).

In further studies, double staining of the retinal sections with an antibody against rhodopsin, the chromophore responsible for light absorbing in the neuroretina of mice, was performed, and the obtained data is presented in FIGS. 3E-3G. As shown, PAR1 co-localized with rhodopsin in rod inner and outer segments.

These findings indicate that PAR1 is overexpressed in the neuroretina in diabetic subjects.

Thrombin activity and lower retinal function in STZ-induced diabetic mice were also tested in extracts of diabetic mice eye posterior segments, according to the procedures described hereinabove.

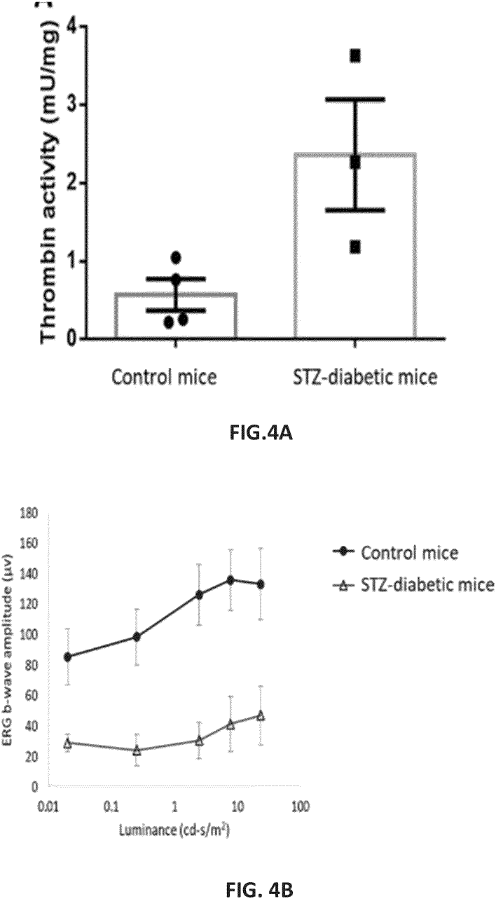

The data are presented in FIGS. 4A-4B. As shown in FIG. 4A, STZ-induced diabetic mice present higher thrombin activity in the posterior segment compared to control, non-diabetic mice. As shown in FIG. 4B, lower retinal function, measured by electroretinography (ERG), is reduced as early as 2 weeks following induction of diabetes in mice by STZ injection.

### Example 3

### Treatment of Diabetes-induced Retinal Degeneration With PARIN5

C57BL/6 mice received either sham (PBS, n=5) or PARIN5 (100 nM in PBS, n=7) treatment for 7 days, starting at three days after diabetes induction by STZ injection. Retinal function was determined by ERG. Treatment included intraperitoneal (IP) injection (0.5 µl/gram, once a day) and eye drops (10 µl/eye, once a day).

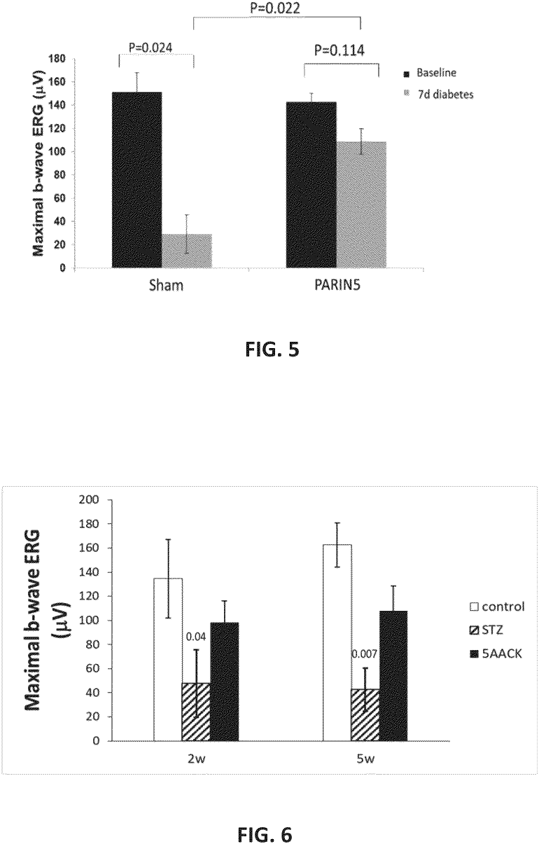

The obtained data are presented in FIG. 5, and show that PARIN5 treatment of STZ-induced diabetic mice preserved mouse retinal function.

In further experiments, STZ-induced diabetic were treated with eye drops, 100 nM PARIN5 in PBS (10 µl/eye, once a day), for 5 weeks. Non-diabetic mice and diabetic non-treated mice served as control.

The obtained data are presented in FIG. 6. Diabetes was induced by STZ injection, resulting in significant deterioration in retinal function as indicated by significantly lower ERG b-wave responses 2 and 5 weeks following STZ injection (p=0.04 and p=0.007, respectively). By contrast, no significant differences were observed in ERG b-wave recordings between control mice and STZ-induced diabetic mice receiving daily eye drops containing PARIN5 (100 nM, all p>0.3).

Notably, the corneal barrier is similar between humans and mice, and it can therefore be predicted that PARIN5 and like molecules will cross the corneal barrier and that PARIN5 eye drops or other ophthalmic formulations can be beneficially used in the treatment of diabetic retinopathy and other conditions that involve neuroretinal degeneration.

Taken together, these studies suggest that PAR1 is expressed in the retina under physiological conditions, and that the PAR1/thrombin plays a role in diabetes-induced retinal degeneration. Furthermore, these findings indicate that retinal function can be preserved and retinal degeneration can be treated and prevented using eye drops containing PAR1 selective inhibitors such as small molecules exemplified by the PARIN5 inhibitor.

These findings support a role for thrombin/PAR1 in DR pathophysiology and progression, and suggest that targeting this pathway presents a promising strategy for treatment of diabetic retinopathy and other neuroretinal degeneration, including the effects on neuroretinal cell loss, retinal structure and function.

### Example 4

### Distribution and Function of PAR1 in Healthy Mice Neuroretina

The expression of PAR1 in the neuroretina was studied by comparing expressions in WT and in knockout PAR1 (PAR1^{-/-}) mice models.

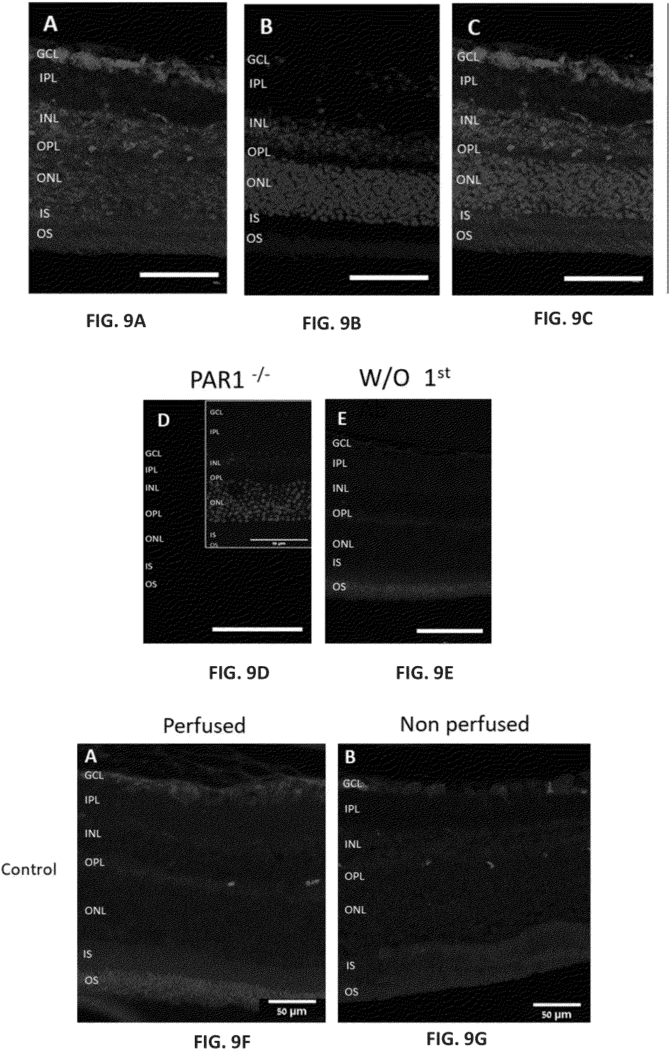

Expression patterns of PAR1 in mice neuroretinas were studied by immunofluorescence, as described under the Materials and Methods section hereinabove. Eyes were enucleated from ten C57BL/6J male mice, paraffin embedded and retinal sections were stained with anti-PAR1 antibody (1:50, Novus Biological). The obtained data is shown in FIGS. 9A-E. As shown in FIGS. 9A-C, staining was observed in the nuclei of retinal ganglion cells, inner nuclear layer cells, and photoreceptors in retinas isolated from WT. No staining was observed in PAR1^{-/-} mice retinas or in the control experiment with a secondary antibody, as seen respectively in FIGS. 9D and 9E. These data suggest that the majority of neuroretinal cells express PAR1 under physiological conditions. Surprisingly, PAR1 staining pattern suggested that the protein is expressed in the nuclei of photoreceptors (outer nuclear layer) and inner nuclear layer cells.

In order to study whether the origin of the PAR1 expressing cells is intrinsic to the retina or extrinsic from blood components, additional C57BL/6J male mice (n=2) were perfused with paraformaldehyde prior to eye enucleation. As can be seen in FIGS. 9F-G, the staining pattern was found to be similar between the non-perfused and the perfused stained slices.

Staining specificity was demonstrated by lack of staining in retinas stained at the same conditions but omitting the primary antibody and by staining retinas from PAR1-/-mice with primary and secondary antibodies.

In order to characterize PAR1 expression pattern in photoreceptors cells, retinas were co-stained with PAR1 and antibodies directed against rhodopsin (1:50, Abcam), that is specifically expressed in rods, M/L-opsin (1:100, Millipore) that is specifically expressed in M/L-cones, and S-opsin (1:100, Millipore) that is expressed in S-cones.

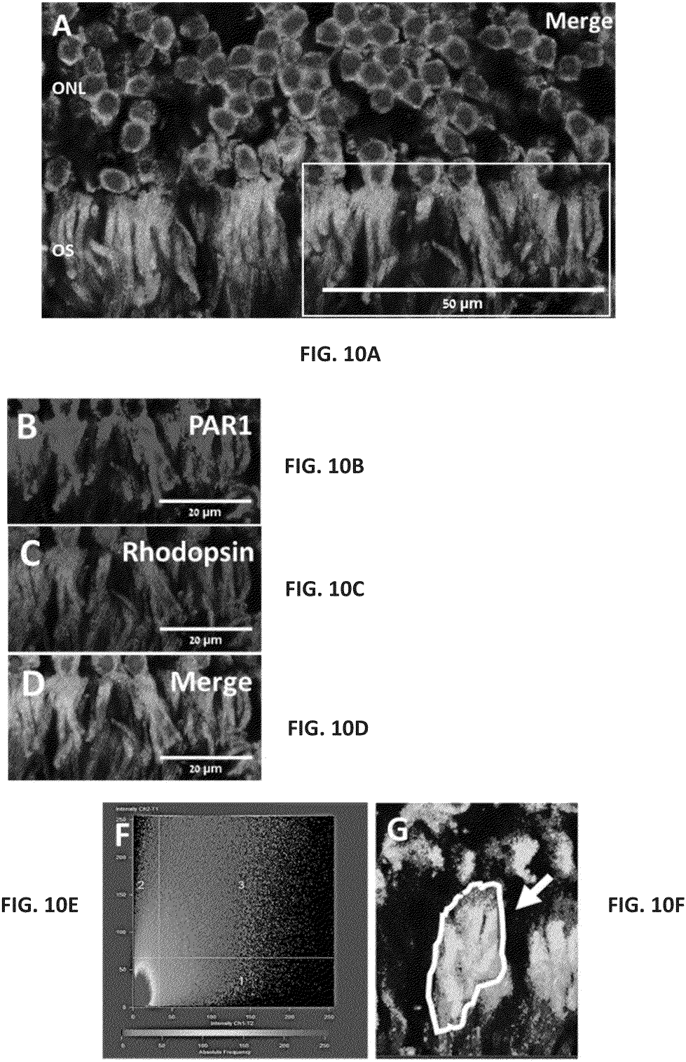

FIGS. 10A-D present the obtained images and indicate that PAR1 is co-localized with rhodopsin in the inner and outer rod cell segments, supporting its expression in rods. Pearson’s Correlation Coefficient between the PAR1 (red channel) and rhodopsin (green channel), shown in FIGS. 10E-F, was 0.92 ± 0.02, indicating a strong overlap of PAR1 and rhodopsin in rod outer and inner segments.

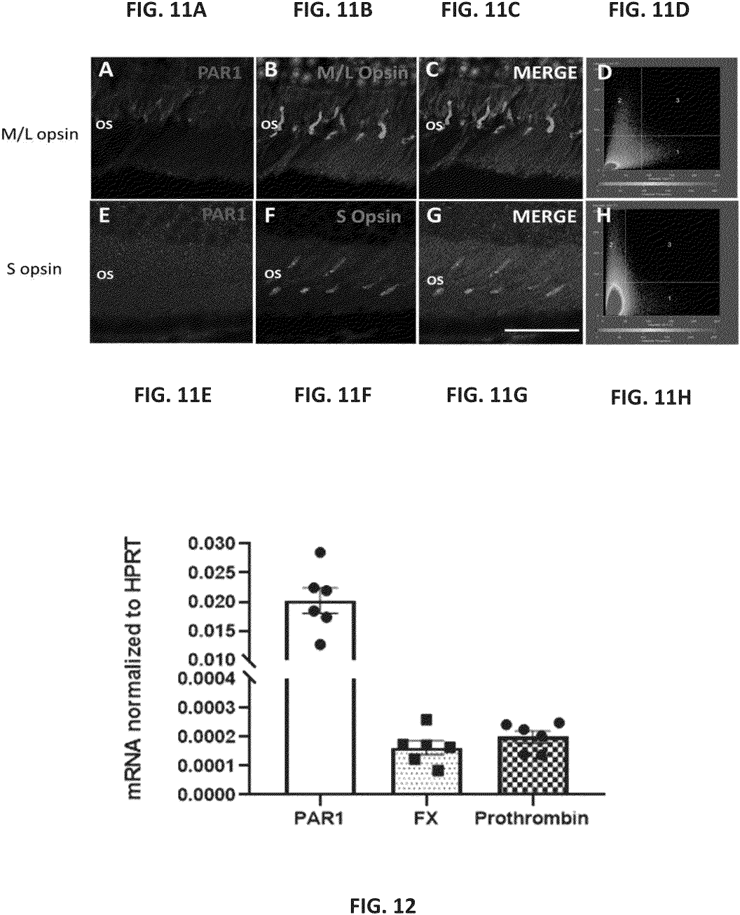

FIGS. 11A-F show PAR1 staining in cone segments. Co-localization analysis showed low correlation between PAR1 and M/L- and S- opsin staining, as presented in FIGS. 11C and 11G, with Pearson Correlation Coefficients of 0.11 ± 0.110 and 0.04 ± 0.01, respectively, depicted in FIGS. 11D-H, indicating weak overlap of PAR1 in cone segments. Thus, no PAR1 staining was observed in M/L and S-cone outer segments.

These data show that PAR1 protein was co-localized with rhodopsin, the light absorbing protein in rod photoreceptors, but not with the M/L- or S- opsin, that mediate light absorption in M/L- and S-cones, respectively. In mice, similar to primates, rods constitute the vast majority (nearly 97 %) of photoreceptors, and cones account for the remainder. Hence these findings support the role for PAR1 in retinal function, and indicate that it may be specifically involved in visual function under low light conditions.

To confirm the staining specificity and further assess the expression of PAR1 protein in the neuroretina, Western blot analysis was performed using the same anti-PAR1 antibody used for the immunofluorescence analysis. This antibody (NBP-71770, Nuvos biologicals) is directed against amino acid sequence Asp35-Arg46 which includes the thrombin cleavage site of PAR1 (Arg41) and the six preceding amino acid residues. Hence, this antibody is predicted to detect mainly the intact PAR1 protein (about 52 kDa).

Neuroretinas and platelets were collected from C57BL/6J WT and PAR1 ^{-/-} mice for Western blot analysis. Previous studies [Arachiche et al. (2014) PLoS One 9, e97724] indicated that PAR1 is not expressed in mouse platelets. Therefore, lysates of mouse platelets were used as a negative control for the Western blot analysis.

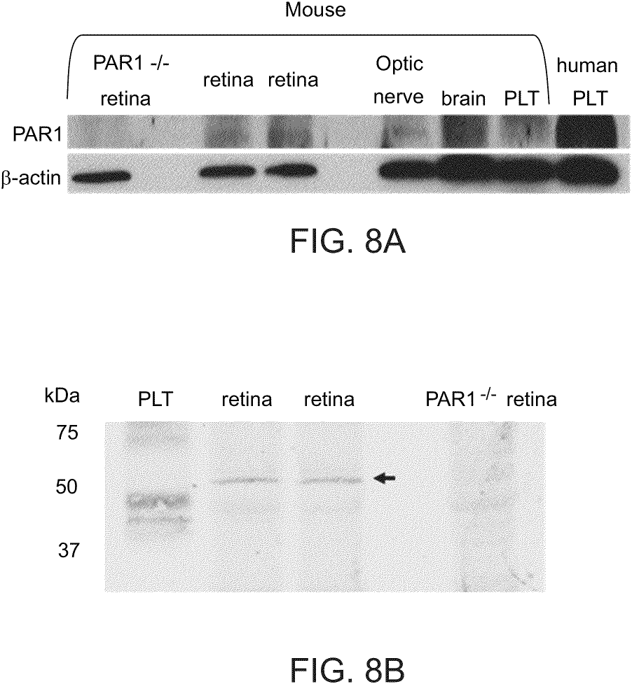

FIG. 8B presents the analysis with PAR1 antibody, demonstrating the specific presence of PAR1 in the mice neuroretina as indicated by a specific band at about 52 kDa recognized by the antibody, but not in mouse platelets or PAR1^{-/-} retina. These data supports the specificity previously indicated by immunofluorescence staining.

As shown in FIG. 8A, additional Western blot analyses on cells lysates from mouse retina, optic nerve and brain demonstrated the antibody specificity and the expression of PAR1 protein (about 75 kDa band) in the mouse neuro-retina. As can be seen, staining was observed in mouse retina, optic nerve and brain and human platelets (PLT), but not in mouse platelet and in PAR1 knockout mice.

qRT-PCR analysis was also used to determine the source of the coagulation proteins and PAR1 in the neuroretina, according to the protocol described hereinabove. The results are presented in FIG. 12 and show that mRNA expression of PAR1, FX, and prothrombin were detected in isolated neuroretinas under physiological conditions. The mRNA expression level of PAR1 was higher by two orders of magnitude in comparison to FX and to prothrombin (0.0202±0.0022, 0.0002±0.0000, 0.0002±0.0000, normalized to HPRT expression, p<0.0005, n=6).

Following the showing of local production of coagulation proteins in the neuroretina, a depolarization experiment was set. KCl-induced depolarization of neuroretinas was performed to test the functional role of the thrombin/PAR1 pathway, as described hereinabove.

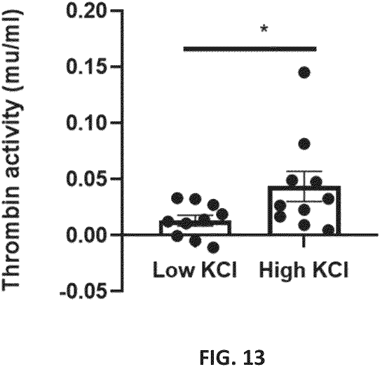

The obtained data are presented in FIG. 13, showing that thrombin activity was increased by nearly 4-fold in mouse neuroretinas following ex-vivo depolarization compared to physiological conditions (0. 044 ± 0.013 mu/ml vs. 0.013 ± 0.005 mu/ml, p=0.0497).

Altogether, these data demonstrate that intrinsic and specific co-localizations of PAR1 and rhodopsin are present in rod photoreceptor outer segments and cell bodies, along with thrombin activity identified during potassium-induced depolarization in the neuroretina, indicating that the PAR1/thrombin pathway is involved in physiological neural function in the retina (neuromodulation of the retina).

### Example 5

### Characterization of PAR1 Expression Pattern in the Neuroretina of DR Human Patients and Mice

Further to uncovering that PAR1 is expressed in the inner and outer neuroretina layers of non-diabetic humans and mice, and that the expression of PAR1 is elevated in mice following diabetes induction and that PAR1 may translocate into the nucleus in the inner and outer retina layers, as described hereinabove, the expression pattern of PAR1 in the neuro-retina of DR human patients was examined.

Neuroretinal cell types which express PAR1 are identified, and double/triple staining immunofluorescence analysis with markers for specific retinal cell types (photoreceptors, bipolar cells, horizontal cells, amacrine cells, Muller cells, ganglion cells and microglial cells) are performed using retinal sections from healthy human donors (n≥3) and DR patients (n≥3) as well as control and STZ-diabetic induced mice at 2, 5 and 10 weeks following diabetic induction (n≥3 each group).

To determine the subcellular localization of PAR1 in healthy conditions and diabetes, immunofluorescence analysis using double staining with specific retinal nuclear markers is performed on mouse and human retina sections, as well as Western blot analysis of healthy and DR mouse neuro-retinas following nuclear and cytoplasmic cell fractionation, in accordance with the procedures described hereinabove.

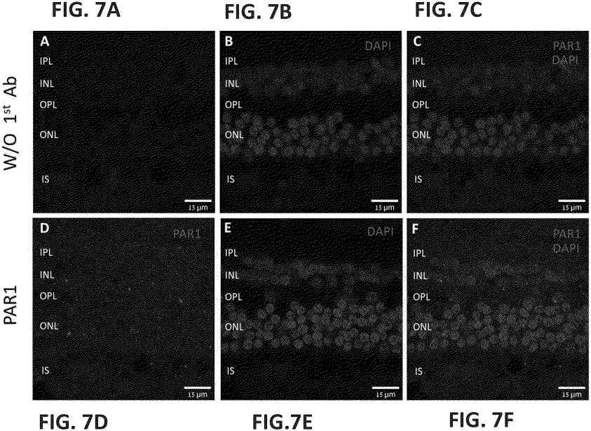

In preliminary studies, paraffin retinal sections from healthy human donors were stained with anti-PAR1 antibody and counter stained with 4′,6-diamidino-2-phenylindole (DAPI). The obtained confocal microscopy images are shown in FIGS. 7A-7F. As shown in FIGS. 7D, 7E and 7F, PAR1 is expressed in retinal inner nuclear layer cells (bipolar cells and amacrine cells), photoreceptor cells and ganglion cells in human eyes under physiological conditions. By contrast, no staining is obtained when the human sections were incubated without PAR1 antibody only with secondary antibody (W/O 1^{st} Ab, FIGS. 7A-C), supporting the staining specificity for the human protein in the retina. These findings indicate that PAR1 is expressed in the human neuro-retina and is overexpressed in the neuroretina of diabetic patients.

To assess the expression pattern of PAR1 in diabetic retina, diabetes was induced in 8 weeks old C57BL/6J male mice (n=12) by a single intraperitoneal injection of 150 mg/Kg STZ (Sigma-Aldrich). Mice were sacrificed 5 weeks following STZ injection. Four mice were perfused with paraformaldehyde before eye enucleation, and retinas from the other 8 mice were removed without perfusion.

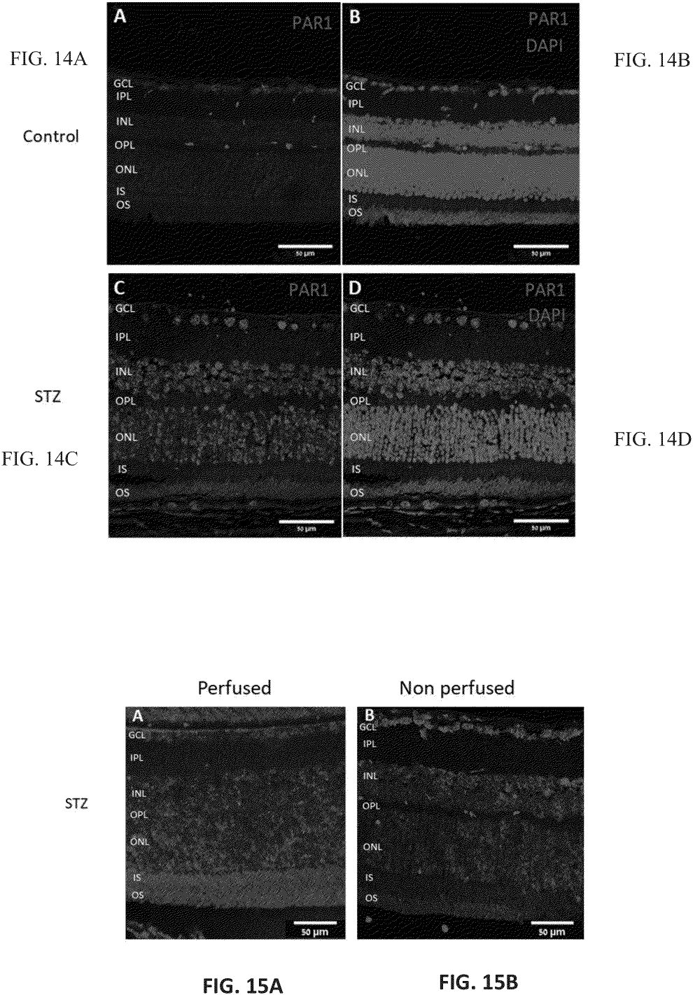

FIGS. 14A-D present the data obtained in immunostaining assays of paraffin retinal sections of non-diabetic (FIGS. 14A-B) and 5-week diabetic (FIGS. 14C-D) C57BL/6 mice stained with anti-PAR1 antibody (red) and counter-stained with DAPI (blue). As can be seen, significantly stronger staining of PAR1 was observed in diabetic mice compared with control mice, with a clear nuclear staining in the inner and outer retinal layers of diabetic mice.

FIGS. 15A-B present the expression pattern in the neuroretina in PFA-perfused vs. non-perfused diabetic mice. Paraffin retinal sections from STZ induced diabetic mice 5 weeks following diabetes induction, were perfused with paraformaldehyde before eye removal (FIG. 15A), and non-perfused diabetic mice (FIG. 15B) were stained with anti-PAR1 antibody (red). As can be seen, there was no significant difference in PAR1 staining pattern between neuroretinas of PFA-perfused and non-perfused mice.

### Example 6

### Treatment Optimization

To elucidate the optimal dosage and regimen of PARIN5-containing eye drops treatment, diabetic mice are treated daily for 2 weeks with eye drops containing PARIN5 at increasing concentrations (0, 10 nM, 100 nM, 1000 nM, n ≥ 17 each group). Mice are evaluated for retinal function by ERG, thrombin activity in the neuroretina and systemic coagulation to determine the optimal efficient and safe treatment dose.

The effect of PARIN5 eye drops on thrombin activity in the retina, and retinal structure and function in a mouse model of DR is assessed by treating diabetic mice daily for 10 weeks with eye drops containing optimal PARIN5 concentration (determined as above) or PBS as control. Mice are assessed, per the methods described hereinabove, at 2, 5 and 10 weeks for: thrombin activity in the neuroretina; retinal structure by multicolor fundus imaging, spectral domain optical coherence tomography (SD-OCT) imaging and histology; retinal function by ERG; apoptosis and inflammation in the neuroretina by immunofluorescence and Western blot analysis; coagulation protein levels in the neuroretina by Western blot analysis; and systemic coagulation.

### Example 7

### Treatment of Retinitis Pigmentosa With PARIN5

Retinitis pigmentosa (RP) is a group of incurable hereditary retinal degeneration diseases affecting one in 4,000 people. These diseases are known to have dozens of causative genes, thereby making genetically diagnosing it impossible for many patients. RP is characterized by progressive degeneration of rod and cone photoreceptors. Photoreceptor degeneration is associated with activation of microglial cells and its migration into the subretina.

The mouse model RPE65/rd12 exhibits RP. By the age of 3 month, it’s cone photoreceptors are lost in the ventral nasal and temporal retina. Degeneration of rods occurs as well, with diminished but recordable ERG.

As it is shown herein that PAR1 is expressed in rod photoreceptors, the treatment of RP in RPE65/rd12 mice was examined using PARIN5.

Neuroretinas were divided into two halves. Each paired-half was placed into a single well in 96-well black microplates (Nunc, Roskilde, Denmark) one in the presence and the other in absence of PARIN5 (100 nM final concentration in PBS). All wells contained a thrombin substrate buffer (in mM: 150 NaCl, 1 CaCl₂, 50 Tris-HCl: pH 8.0), bovine serum albumin (BSA, 0.1 %, 9048-46-8, Amresco, Ohio), bestatin (0.1 mg/ml, 70520, Cayman-chemical company, Michigan) and prolylendopeptidase inhibitor (0.2 mM, 537011, Calbiochem, San Diego).

Thrombin enzymatic activity was measured using a fluorometric assay, based on the cleavage rate of the synthetic thrombin fluorogenic substrate Boc-Asp (OBzl)-Pro-Arg-AMC (14 µM, I-1560, Bachem, Switzerland) as previously described (Gera et al. 2016, supra). The fluorescence signal was measured by a microplate reader (Infinite 2000; Tecan, Männedorf, Switzerland) with excitation and emission filters of 360 ± 35 nm and 460 ± 35 nm, respectively. A calibration curve was used in each experiment with 0.00078-0.05 u/ml bovine thrombin (T-4648, Sigma-Aldrich).

The obtained data are shown in FIG. 16. As can be seen, a significant decreased thrombin activity was measured in the RPE65/rd12 mice compare to wild-type C57BL mice. PARIN5 significantly inhibited both basal and decreased thrombin activity.

The basal thrombin activity in neuroretinas isolated from WT mice was inhibited by PARIN5 (0.12 ± 0.013 mU/ml vs. 0.007 ± 0.005 mU/ml respectively, p<0.0001) (circle-shaped). A 2-fold decrease in thrombin activity was measured in neuroretinas isolated from RPE65/rd12 mice (squares; 0.06 ± 0.028 mU/ml, p=0.0153) compared to the WT controls (circles). The thrombin activity in neuroretinas isolated from RPE65/rd12 mice significantly decreased further in the presence of 100 nM PARIN5 (0.003 ± 0.002 mU/ml, p=0.0178) (squares).

These findings further demonstrate the inhibitory effect of PARIN5 on the neuroretina, further supporting a role of this and like molecules in the treatment of retinal degeneration disorders.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1. An ophthalmic formulation comprising a PAR1 antagonist and/or an agent that interferes with an interaction of PAR1 and a protease, and an ophthalmically acceptable carrier.

2. The ophthalmic formulation of claim 1, wherein said agent is represented by Formula I: 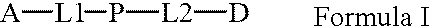
wherein:
P is a peptide of at least 3 amino acid residues, comprising or consisting of the amino acid sequence Asp-Pro-Arg;
A is an N-terminus protecting group;
D is a group capable of interfering with a PAR1/protease interaction; and
L1 and L2 are each independently a linking moiety or absent,
and a pharmaceutically acceptable carrier, the formulation being for topical application of said agent to an eye of a subject in need thereof.

3. The ophthalmic formulation of claim 2, wherein P in Formula I consists of said amino acid sequence Asp-Pro-Arg.

4. The ophthalmic formulation of claim 2, wherein P in Formula I has 5 amino acid residues.

5. The ophthalmic formulation of claim 4, wherein P in Formula I has an amino acid sequence as set forth in SEQ ID NO:2 (TLDPR).

6. The ophthalmic formulation of claim 2, wherein P in Formula I has an amino acid sequence selected from the amino acid sequences as set forth in SEQ ID NOS:1-17.

7. The ophthalmic formulation of claim 2, wherein A in Formula I is an aromatic N-terminus protecting group.

8. The ophthalmic formulation of claim 2, wherein D in Formula I is a protease inhibitor.

9. The ophthalmic formulation of claim 8, wherein D in Formula I is a thrombin inhibitor.

10. The ophthalmic formulation of claim 2, wherein D in Formula I is or comprises an acetyl group.

11. The ophthalmic formulation of claim 2, wherein D in Formula I is or comprises chloromethyl ketone.

12. The ophthalmic formulation of claim 1, being configured for topical application to an eye of a subject.

13. The ophthalmic formulation of claim 1, being in a form of an aqueous solution.

14. The ophthalmic formulation of claim 1, wherein a concentration of said PAR1 antagonist and/or said agent is lower than 500 millimolar, or lower than 100 millimolar, or lower than 1 millimolar, or lower than 500 nanomolar, or lower than 100 nanomolar, or lower than 1 nanomolar.

15. A method of treating a disease or disorder associated with overexpression and/or overactivity of PAR1 in a retinal tissue of a subject and/or a disease or disorder treatable by interfering with a PAR1/protease interaction in a retinal tissue of a subject, the method comprising topically administering the ophthalmic formulation to an eye of the subject or contacting the ophthalmic formulation of claim 1 with the eye of the subject.

16. The method of claim 15, wherein said disease or disorder is selected from retinal degeneration, retinal dystrophy, retinal inflammation and abnormal proliferation in the retinal tissue.

17. A method of treating or preventing retinal degeneration in a subject in need thereof, the method comprising topically administering the ophthalmic formulation to an eye of the subject or contacting the ophthalmic formulation of claim 1 with the eye of the subject.

18. The method of claim 17, wherein said retinal degeneration is associated with diabetic retinal neuropathy.

19. The method of claim 15, wherein said treating comprises topically administering the formulation to the eye of the subject from 1 to 4 times per day.

20. An article-of-manufacturing comprising the ophthalmic formulation of claim 1 and means for topically administering the formulation to, or contacting the formulation with, an eye of a subject.
